(19) United States
(12) Patent Application Publication
(10) Pub. No.: US 2009/0228995 A1
(43) Pub. Date: Sep. 10, 2009

(54) Polymorphisms and Haplotypes of the Alpha 2C Adrenergic Receptor Gene

(76) Inventors: Stephen Bryant Liggett, Cincinnati, OH (US); Kersten M. Small, Cincinnati, OH (US)

Correspondence Address: FENWICK & WEST LLP, SILICON VALLEY CENTER, 801 CALIFORNIA STREET, MOUNTAIN VIEW, CA, 94041, US

(21) Appl. No.: 11/571,151

(22) PCT Filed: Jun. 29, 2005
(86) PCT No.: PCT/US05/23293

§ 371 Date: May 13, 2009

Related U.S. Application Data
(60) Provisional application No. 60/583,846, filed on Jun. 29, 2004.

(51) Int. Cl. A01K 67/027; C12N 15/11; C12N 15/00; C07K 14/00; C12N 5/06; C12Q 1/68

(52) U.S. Cl. 800/13; 536/23.5; 435/320.1; 530/350; 435/325; 435/6

(57) ABSTRACT

Methods and compositions for diagnosing and treating diseases are provided. The methods involve the discovery of a correlation between an α2C-adrenergic receptor gene polymorphism, a combination of polymorphisms or a haplotype and the occurrence of diseases such as heart failure, cardiac arrhythmias, hypertension, behavioral and learning disorders, psychiatric diseases such as depression, obesity, and diabetes mellitus. The invention further pertains to the use of such molecules and methods in the diagnosis, prognosis, and treatment selection for diseases such as heart failure, cardiac arrhythmias, hypertension, behavioral and learning disorders, psychiatric diseases such as depression, obesity, and diabetes mellitus. The invention also pertains to a composition of matter comprising polymorphisms and haplotypes of the α2C-adrenergic receptor.

### CROSS REFERENCE TO RELATED APPLICATIONS

This application claims the benefit of U.S. Provisional Application No. 60/583,646, filed Jun. 29, 2004, the entire disclosure of which is hereby incorporated by reference in its entirety for all purposes.

### STATEMENT REGARDING FEDERALLY SPONSORED RESEARCH OR DEVELOPMENT

The U.S. Government has certain rights in this invention pursuant to Grant No. HL52318 awarded by the National Institutes of Health.

### BACKGROUND OF THE INVENTION

1. Field of the Invention

The invention pertains to polymorphisms and haplotypes of the alpha 2C adrenergic receptor (α_{2C}AR) gene. Nucleic acids and polypeptides are disclosed. The invention further pertains to the use of such molecules in kits and methods for diagnosis, prognosis, and treatment selection of conditions related to α_{2C}AR, such as heart failure.

2. Description of the Related Art

The adrenergic receptors, whose endogenous ligands are epinephrine and norepinephrine, are members of the superfamily of 7-transmembrane spanning G-protein coupled receptors. Adrenergic receptors modulate a host of functions relative to the sympathetic nervous system, including neurotransmitter release, and cardiac, vascular, pulmonary, renal, metabolic and central nervous system function. For over a decade it has been known that the expression and/or function of adrenergic receptors varies considerably between individuals (1), even when studies are carried out under stringent conditions in normal individuals. Similarly, the physiologic or clinical response to receptor agonists and antagonists shows marked interindividual variability (2, 3). Evidence has accumulated with some of the adrenergic receptors over the last few years suggesting that interindividual differences in receptor expression, function or patient's response to treatment aimed at these receptors is based on genetic variability of the genes encoding these receptors (4).

Of recent interest has been genetic variability of the α2-adrenergic receptors (α2ARS) (5-7). The α_{2A}- and α_{2C}AR subtypes are localized to presynaptic nerve terminals and participate in a negative feedback loop regulating norepinephrine release (8). In settings of high sympathetic activity these subtypes serve to dampen further norepinephrine release. One such setting is human heart failure of virtually all etiologies, where marked sympathetic drive develops as a response to low cardiac output and systemic perfusion (9). However, persistent stimulation by norepinephrine of post-synaptic β1AR expressed on cardiomyocytes leads to multiple deleterious signaling events in the heart, ultimately leading to a worsening of pump function and clinical deterioration (9, 10).

The critical roles of the α2ARs in modulating norepinephrine release and its cardiac consequences have been delineated in mice lacking one or both of these subtypes. α2A/α2C double knockout mice develop a catecholamine-mediated dilated cardiomyopathy, indicating the necessity of this mechanism for regulation of norepinephrine release under non-stressed conditions (8). In addition, α_{2C}AR−/− mice develop a lethal cardiomyopathy when subjected to pressure overload via aortic banding (7), which is consistent with uncontrolled norepinephrine release from cardiac presynaptic nerves acting to persistently stimulate the pressure-compromised heart.

It is also known that the α_{2C}AR is a critical receptor in the central nervous system, and modulates release of norepinephrine and other neurotransmitters. The α_{2C}AR appears to play important roles in regulating behavioral, learning and cognitive functions of the central nervous system. These include cortical electroencephalogram arousal, the startle reflex, isolation-induced aggression, and stress-induced despair response (11).

Thus a genetic polymorphism of the human α_{2C}AR gene which leads to modulated expression or function, might act to predispose individuals to heart failure, or to potential failure due to other causes such as myocardial infarction or hypertension. Further, a functional genetic variation of the α_{2C}AR gene may affect predisposition to neuropsychiatric and behavioral and learning syndromes or the response to pharmacologic treatment (11).

To date, a single α_{2C}AR polymorphism has been identified. Located within the third intracellular loop of α_{2C}AR, the polymorphism consists of an in-frame 12 nucleotide deletion resulting in loss of Gly-Ala-Gly-Pro from this G-protein coupling domain of the receptor (12). In CHO cells recombinantly expressing wild-type and this polymorphic α_{2C}AR (denoted α2CDel322-325), the variant receptor has substantially decreased coupling to its cognate G-protein Gi, with decreased inhibition of adenylyl cyclase and decreased stimulation of mitogen activated protein kinase (12). The polymorphism has been associated with increased cardiac 125I-MIBG (a radiolabeled norepinephrine analog) imaging in heart failure patients (6), earlier onset of heart failure (5), and heart failure severity or progression (5, 7). In addition, it has been shown (5) that the α2CDel322-325 is an independent risk factor for the development of heart failure in African-Americans (odds ratio ˜4 compared to healthy African-Americans), and that such risk may be synergistic with a hyperfunctional β1AR variant.

In the aforementioned studies, it has been recognized that even in α2_{C}Del322-325 groups, there is noticeable variability in the phenotype. Furthermore, this polymorphism is present at a high frequency only in populations of African descent.

Because of the central role of the α_{2C}AR in regulating norepinephrine release, the genetic variability of this gene likely plays a prominent role in cardiomyopathy regardless of ethnicity. It would therefore be useful to identify other polymorphisms in the α_{2C}AR gene.

Given the importance of α_{2C}AR in modulating a variety of physiological functions, there is a need in the art for improved methods to identify α_{2C}AR polymorphisms and to correlate the identity of these polymorphisms with the other functions of α-adrenergic receptors. The polymorphisms could be used in methods useful for the diagnosis, prognosis, and treatment selection of α_{2C}AR related conditions such as, e.g., heart failure, cardiac arrhythmias, hypertension, behavioral and learning disorders, psychiatric diseases such as depression, obesity, and diabetes mellitus.

### SUMMARY OF THE INVENTION

The present invention provides polymorphisms, e.g., polymorphic sites, and haplotypes, e.g., variants, of the α2C adrenergic receptor (α_{2C}AR) gene. Described herein is identification of 23 polymorphic sites representing 30 haplotypes as described in SEQ ID NOS:1-30. Also described is the functional impact each α_{2C}AR variant has on expression of the receptor in cells. Provided are nucleic acids, probes, primers, kits, and genotyping methods useful for the diagnosis, prognosis, and treatment selection for α_{2C}AR related conditions.

One embodiment of the invention encompasses an isolated polynucleotide comprising a sequence of an α_{2C}AR variant, or a complement thereof, wherein the sequence consists of one of SEQ ID NOS:1-30. The polynucleotides can be DNA or RNA, and can be double- or single-stranded.

Another embodiment encompasses an isolated polynucleotide comprising a sequence of an α_{2C}AR variant, or a complement thereof, wherein the sequence comprises at least one nucleotide variation located at an α_{2C}AR polymorphic site, wherein an α_{2C}AR polymorphic site is nucleotide 59, 222, 281, 358, 569, 574, 712, 946, 1125, 1376, 1673, 1698, 1705, 1942, 2397, 2408, 3570, 3633, 3804, 4110-4130, 4123, or 4394. In some embodiments, the polynucleotides described comprise 2, 3, 4, 5, 6, 7, 8, 9, 10, 11, 12, 13, 14, 15, 16, 17, 18, 19, 20, 21, or 22 nucleotide variations. Other embodiments further comprise a deletion of α_{2C}AR nucleotides 3601-3612 (e.g., α_{2C}AR Del322-325).

In addition, the invention provides vectors, libraries, cells, and transgenic animals comprising the polynucleotides of the invention. Also included in the invention are isolated α_{2C}AR variant polypeptides encoded by the α_{2C}AR polynucleotides of the invention. Further embodiments include antibodies specific for α_{2C}AR variant polypeptides.

The invention further provides oligonucleotides that specifically hybridize to a sequence shown in SEQ ID NOS:1-30, or its complement. These oligonucleotides can be probes or primers.

The invention also provides kits suitable for, e.g., genetic testing, comprising at least one probe for detecting at least one α_{2C}AR polymorphic site. The kit can comprise probes for detecting more than one α_{2C}AR polymorphic site. In other embodiments the kit comprises probes for detecting enough α_{2C}AR polymorphic sites to assign a α_{2C}AR haplotype. In preferred embodiments, the kit also comprises at least one probe for detecting a polymorphism at α_{2C}AR nucleotides 3601-3612.

Also disclosed are methods for detecting at least one α_{2C}AR polymorphic site. In some embodiments the methods further comprise detecting a polymorphism at α_{2C}AR nucleotides 3601-3612.

Also contemplated are methods for α_{2C}AR genotyping of an individual. The method includes the steps of a) obtaining at least one sample from the individual; b) detecting at least one α_{2C}AR polymorphic site in the sample and c) comparing the identity of the at least one polymorphic site with a known data set. In some embodiments, the method further comprises detecting a polymorphism at α_{2C}AR nucleotides 3601-3612. In other embodiments, the method further comprises a step related to prognosis of, diagnosis of, or treatment selection for a condition associated with α_{2C}AR, e.g., heart failure.

The present invention also provides a computer system for storing and displaying polymorphism data determined for the α_{2C}AR gene. In another embodiment, the invention provides a computer readable medium having stored thereon a sequence selected from the group consisting of a nucleic acid code comprising a contiguous span of at least 12 nucleotides of any of SEQ ID NOS:1-30, or the complements thereof.

Other embodiments and advantages of the invention are set forth in the following detailed description and claims.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a visual depiction of the organization of α_{2C}AR polymorphisms into haplotypes stratified by ethnic group. In a, b, c each column represents a polymorphism location as labeled, and each horizontal row represents one of the 105 individuals in the cohort (not labeled). Homozygosity for the reference allele is colored blue, heterozygosity is red, and homozygosity for the polymorphism is yellow. Gray indicates that in the presence of the homozygous deletion at position r, the s genotype is not applicable. In d, e, f the degree of linkage disequilibrium (A) between any two polymorphisms is shown (note scale). White indicates that the genotype data is non-informative in the indicated cohort and thus linkage disequilibrium is indeterminate. Ca, Caucasian; AA, African-American; As, Asian.

FIG. 2 illustrates that α_{2C}AR haplotypes display differential mRNA expression profiles. Neuronal BE(2)-C cells were transfected with the indicated haplotype constructs and α_{2C}AR transcripts quantitated by real-time RT-PCR. Data represent mean±standard error of 6 independent experiments. α_{2C}AR mRNA was significantly related to haplotype (P<0.001 by ANOVA).

FIG. 3 illustrates that α_{2C}AR haplotypes display differential receptor protein expression profiles. Neuronal BE(2)-C cells were transfected with the indicated haplotype constructs and α_{2C}AR protein expression was quantitated by [³H]yohimbine radioligand binding. Data represent mean±standard error of 9 independent experiments. α_{2C}AR protein expression was significantly related to haplotype (P<0.001 by ANOVA).

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

### DETAILED DESCRIPTION OF THE INVENTION

The α2C-adrenergic receptor plays an important role in regulating a variety of physiological functions. The present invention reveals a highly variable α_{2C}AR gene with complex intragenic organization of 23 polymorphic sites representing 30 haplotypes described in SEQ ID NOS:1-30. This includes a common 3′UTR substitution SNP within an insertion/deletion sequence, a radical coding polymorphism that deletes four amino acids, relatively low linkage disequilibrium between many polymorphisms, few cosmopolitan haplotypes and prevalent ethnic-specific haplotypes, substantial divergence between certain haplotype pairs, and partitioning of the previously described cardiomyopathic Del322-325 polymorphism into multiple haplotypes. These haplotypic variations have biological relevance as assessed in studies of receptor transcript and protein expression.

Preferred α_{2C}AR polymorphisms are listed in Table 4, which also includes α_{2C}AR Del322-325. Haplotypes are described in SEQ ID NOS:1-30. The invention includes polynucleotides comprising at least one α_{2C}AR polymorphism, and methods and kits for using the polynucleotides in the diagnosis, prognosis, and treatment selection for α_{2C}AR-related conditions.

### DEFINITIONS

Terms used in the claims and specification are defined as set forth below unless otherwise specified.

It must be noted that, as used in the specification and the appended claims, the singular forms “a,” “an” and “the” include plural referents unless the context clearly dictates otherwise.

“α_{2C}AR gene” is the alpha 2C adrenergic receptor gene. For reference purposes only, GenBank accession numbers NM_{—}000683 and AF280399 (herein incorporated by reference) are examples of sequences reported as human α_{2C}AR gene sequence. Disclosed herein is SEQ ID NO:1, with GenBank accession number AY605898 (herein incorporated by reference).

“α_{2C}AR variant” is a sequence of an α_{2C}AR gene, e.g., as described in SEQ ID NOS:1-30.

“α_{2C}AR polymorphic site” is one of the 23 polymorphic sites identified in the examples below and listed in Table 3 as a through p and r through w; the sites are at α_{2C}AR nucleotide locations 59, 222, 281, 358, 569, 574, 712, 946, 1125, 1376, 1673, 1698, 1705, 1942, 2397, 2408, 3570, 3633, 3804, 4110-4130, 4123, or 4394. The term α_{2C}AR polymorphic site, as described herein, does not include the previously described deletion mutant at α_{2C}AR nucleotides 3601-3612 (e.g., α_{2C}AR Del322-325). For the purposes of identifying the location of a polymorphic site, the first nucleotide of SEQ ID NO:1 is considered nucleotide 1. To further clarify the location of the nucleotide in the gene, in some cases (such as in Table 3), the nucleotides are shown in relation to the start codon of the coding region (the adenine of the ATG in a DNA molecule and the adenine of the AUG in an RNA molecule) of the α_{2C}AR gene which is considered nucleotide “1.” Similarly, the first amino acid of the translated protein product (the methionine) is considered amino acid “1.”

“α_{2C}AR related condition” refers to a variety of diseases or conditions, the susceptibility to which can be indicated in a subject based on the identification of one or more polymorphic sites within the α_{2C} receptor. Examples include but are not limited to cardiovascular diseases such as heart failure, cardiac arrhythmias, and hypertension; behavioral and learning disorders, psychiatric diseases such as depression, obesity, and diabetes mellitus.

“Antibody” as used herein refers to a binding agent including a whole antibody or a binding fragment thereof which is specifically reactive with a polypeptide, e.g., with an α_{2C}AR variant polypeptide or fragment thereof. Antibodies can be fragments, e.g., Fabs. As used herein, the term includes bispecific, single-chain, chimeric, and humanized molecules having affinity for an α_{2C}AR variant polypeptide.

“Cells,” “host cells” or “recombinant host cells” are terms used interchangeably herein to refer not only to the particular subject cell but to the progeny or potential progeny of such a cell. Because certain modifications may occur in succeeding generations due to either mutation or environmental influences, such progeny may not, in fact be identical to the parent cell, but still are included within the scope of the term as used herein.

“Haplotype” as used herein is intended to refer to a set of alleles that are inherited together as a group (i.e., are in linkage disequilibrium) at statistically significant levels (P_{corr}<0.05).

“Isolated” as used herein with respect to nucleic acids or polypeptides, refers to molecules separated from at least one other nucleic acid or polypeptide, respectively, that is present in the natural source of the macromolecule. The term isolated as used herein also refers to a nucleic acid or polypeptide that is substantially free from other nucleic acids or polypeptides that are present in the natural source of the macromolecule, or that is substantially free of cellular material, viral material or culture medium when produced by recombinant DNA techniques, or chemical precursors or other chemicals when chemically synthesized.

“Oligonucleotide” as used herein is defined as a polynucleotide comprised of less than about 100 nucleotides.

“Polymorphism” refers to the coexistence in a population of more than one form of a gene or portion (e.g., allelic variant) thereof. A portion of a gene of which there are at least two different forms, i.e., two different nucleotide sequences, is referred to as a polymorphic site. A specific genetic sequence at a polymorphic site is an allele. A polymorphic site can be a single nucleotide, the identity of which differs in different alleles. A polymorphic site can also be several nucleotides long. A polymorphism is thus then said to be “allelic,” in that, due to the existence of the polymorphism, some members of a population carry a gene with one sequence, whereas other members carry a second, slightly different sequence. In the simplest case, only one variant of the sequence may exist, and the polymorphism is said to be diallelic. The occurrence of alternative mutations can give rise to triallelic polymorphisms, etc. An allele may be referred to by the nucleotide(s) that comprise the sequence difference.

“Polynucleotide” refers to nucleic acids such as deoxyribonucleic acid (DNA) and ribonucleic acid (RNA). The term should also be understood to include, as equivalents, analogs of either RNA or DNA made from nucleotide analogs (e.g., peptide nucleic acids) and as applicable to the embodiment being described, single (sense or antisense) and double-stranded polynucleotides. The term “nucleotide” as used herein is intended to refer to ribonucleotides, deoxyribonucleotides, acyclic derivatives of nucleotides, and functional equivalents thereof, of any phosphorylation state. Functional equivalents of nucleotides are those that act as substrates for a polymerase as, for example, in an amplification method. Functional equivalents of nucleotides are also those that may be formed into a polynucleotide that retains the ability to hybridize in a sequence specific manner to a target polynucleotide.

“Transgenic animal” refers to any animal, preferably a non-human mammal, e.g., a mouse, in which one or more of the cells of the animal contains heterologous nucleic acid introduced by way of human intervention, such as by transgenic techniques well known in the art.

“Treating” as used herein is intended to encompass an approach for curing as well as ameliorating at least one symptom of a condition or disease.

“Vector” refers to a nucleic acid molecule that is capable of transporting another nucleic acid to which it has been linked.

Polynucleotides of the Invention

In one embodiment, the invention provides an isolated polynucleotide comprising a sequence of a variant of the α_{2C}AR gene as disclosed in SEQ ID NOS:1-30. Alternatively, the invention provides a α_{2C}AR polynucleotide that contains at least one of the novel polymorphic sites described herein, see, e.g., Table 3. Thus, the invention specifically does not include polynucleotides comprising a nucleotide sequence identical to other reported α_{2C}AR sequences or to portions thereof, except for genotyping oligonucleotides as described herein.

One embodiment of the invention encompasses an isolated polynucleotide comprising a sequence of an α_{2C}AR variant, wherein the sequence consists of one of SEQ ID NOS:1-30.

Another embodiment encompasses an isolated polynucleotide comprising a sequence of an α_{2C}AR variant, or a complement thereof, wherein the sequence comprises at least one nucleotide variation located at an α_{2C}AR polymorphic site, wherein an α_{2C}AR polymorphic site is nucleotide 59, 222, 281, 358, 569, 574, 712, 946, 1125, 1376, 1673, 1698, 1705, 1942, 2397, 2408, 3570, 3633, 3804, 4110-4130, 4123, or 4394. In some embodiments, the polynucleotides described comprise 2, 3, 4, 5, 6, 7, 8, 9, 10, 11, 12, 13, 14, 15, 16, 17, 18, 19, 20, 21, or 22 nucleotide variations. Other embodiments further comprise a deletion of α_{SC}AR nucleotides 3601-3612.

Also included in the scope of the invention are polynucleotides and complements thereof having at least 95% homology to an α_{2C}AR variant. In some embodiments, the polynucleotides comprise at least 96% or at least 97% or at least 98% or at least 99% homology to an α_{2C}AR variant.

Also contemplated are α_{2C}AR polynucleotide fragments, e.g., fragments of an α_{2C}AR polynucleotide that contains at least one of the novel polymorphic sites described herein. A polynucleotide fragment can be between, e.g., 10-4000 basepairs (bp), in length. In some embodiments, the α_{2C}AR polynucleotide fragments are between, e.g., 10-1000 bp or 10-500 bp or 10-250 bp or 10-100 bp or 10-80 bp or 20-80 bp in length.

Polynucleotides of the invention can be either DNA or RNA, single-stranded or double-stranded. In one embodiment, the polynucleotides are single-stranded DNA. Such molecules may also be fragments, portions, and segments thereof and molecules, such as oligonucleotides, that specifically hybridize to an α_{2C}AR gene nucleic acid molecule. Such molecules may be isolated, derived, or amplified from a biological sample. Alternatively, the molecules of the present invention may be chemically synthesized.

In describing the polymorphic sites identified herein, reference is made to the sense strand of the gene for convenience. However, as recognized by the skilled artisan, nucleic acid molecules containing the α_{2C}AR gene may be complementary double-stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the α_{2C}AR genomic variants described herein.

Polymorphic variants of the invention may be prepared by isolating a clone containing the α_{2C}AR gene from a human genomic library. The clone may be sequenced to determine the identity of the nucleotides at the polymorphic sites described herein. Any particular variant claimed herein could be prepared from this clone by performing in vitro mutagenesis using procedures well-known in the art. Alternatively, a polymorphic variant of the α_{2C}AR gene may be chemically synthesized.

The α_{2C}AR haplotypes may be isolated using any method that allows separation of the two “copies” of the α_{2C}AR gene present in an individual, which, as readily understood by the skilled artisan, may be the same allele or different alleles. Separation methods include targeted in vivo cloning (TIVC) in yeast as described in WO 98/01573, U.S. Pat. No. 5,866,404, and U.S. application Ser. No. 08/987,966. Another method, which is described in U.S. application Ser. No. 08/987,966, uses an allele specific oligonucleotide in combination with primer extension and exonuclease degradation to generate hemizygous DNA targets. Yet other methods are single molecule dilution (SMD) as described in Ruano et al., Proc. Natl. Acad. Sci. 87:6296-6300, 1990; and allele specific PCR (Ruano et al., 17 Nucleic Acids. Res. 8392, 1989; Ruano et al., 19 Nucleic Acids Res. 6877-6882, 1991; Michalatos-Beloin et al., 24 Nucleic Acids Res. 4841-4843, 1996).

The invention also provides α_{2C}AR genome anthologies, which are collections of α_{2C}AR haplotypes found in a given population. The population may be any group of at least two individuals, including but not limited to a reference population, a population group, a family population, a clinical population, and a same sex population. An α_{2C}AR genome anthology may comprise individual α_{2C}AR haplotypes stored in separate containers such as micro-test tubes, separate wells of a microtitre plate and the like. Alternatively, two or more groups of the α_{2C}AR haplotypes in the anthology may be stored in separate containers.

The invention also provides for vectors comprising the polynucleotides of the invention. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops, which in their vector form are not bound to the chromosome. In the present specification, “plasmid” and “vector” are used interchangeably as the plasmid is the most commonly used form of vector. However, the invention is intended to include such other forms of expression vectors which serve equivalent functions and which currently are or subsequently become known in the art.

Oligonucleotides

One aspect of the invention includes oligonucleotides useful as probes and primers for identification and detection of α_{2C}AR polymorphisms. Oligonucleotides of the invention specifically hybridize with a nucleic acid molecule comprising an α_{2C}AR sequence.

Oligonucleotides can be used as probes of a nucleic acid sample, such as genomic DNA, mRNA, or other suitable sources of nucleic acid. For such purposes, the oligonucleotides must be capable of specifically hybridizing to a target polynucleotide, e.g., an α_{2C}AR nucleic acid molecule. As used herein, two nucleic acid molecules are said to be capable of specifically hybridizing to one another if the two molecules are capable of forming an anti-parallel, double-stranded nucleic acid structure under hybridizing conditions, whereas they are substantially unable to form a double-stranded structure when incubated with a non-α_{2C}AR nucleic acid molecule under the same conditions.

Thus, for an oligonucleotide to serve as a probe, it must generally be complementary in sequence and be able to form a stable double-stranded structure with a target polynucleotide under the particular environmental conditions employed. The term “allele-specific oligonucleotide” refers to an oligonucleotide probe that is able to hybridize to a region of a target polynucleotide spanning the sequence, mutation, or polymorphism being detected and is substantially unable to hybridize to a corresponding region of a target polynucleotide that either does not contain the sequence, mutation, or polymorphism being detected or contains an altered sequence, mutation, or polymorphism. As will be appreciated by those in the art, allele-specific is not meant to denote an absolute condition. Allele-specificity will depend upon a variety of environmental conditions, including salt and formamide concentrations, hybridization and washing conditions and stringency. Depending on the sequences being analyzed, one or more allele-specific oligonucleotides may be employed for each target polynucleotide. Preferably, allele-specific oligonucleotides will be completely complementary to the target polynucleotide. However, departures from complete complementarity are permissible, e.g., oligonucleotide probes that are substantially complementary are included in the invention.

A nucleic acid molecule is said to be “completely complementary” to a target polynucleotide if, e.g., every nucleotide of one of the molecules is complementary to a nucleotide of the other. In general, complementary molecules can hybridize to one another with sufficient stability to permit them to remain annealed to one another under conventional “high-stringency” conditions.

Two molecules are said to be “substantially complementary” if they can hybridize to one another with sufficient stability to permit them to remain annealed to one another under at least conventional “low-stringency” conditions. Conventional stringency conditions are described, for example, by Sambrook, J., et al., (In: Molecular Cloning, a Laboratory Manual, 2nd Edition, Cold Spring Harbor Press, Cold Spring Harbor, N.Y. (1989)), and by Haymes, B. D., et al. (In: Nucleic Acid Hybridization, A Practical Approach, IRL Press, Washington, D.C. (1985)), both herein incorporated by reference).

Substantially complementary oligonucleotide probes are permissible, as long as such departures do not completely preclude the capacity of the molecules to form a double-stranded structure. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith for the purposes employed. However, for detection purposes, particularly using labeled sequence-specific probes, the primers typically have exact complementarity to obtain the best results.

In order for an oligonucleotide to serve as a primer oligonucleotide, however, it typically need only be sufficiently complementary in sequence to be able to form a stable double-stranded structure under the particular environmental conditions employed. Establishing environmental conditions typically involves selection of solvent and salt concentration incubation temperatures and incubation times. The terms “primer” or “primer oligonucleotide” as used herein refer to an oligonucleotide as defined herein, which is capable of acting as a point of initiation of synthesis when placed under conditions in which synthesis of a primer extension product which is complementary to a nucleic acid strand is induced, as for example, in a PCR reaction. As with non-primer oligonucleotides, primer oligonucleotides may be labeled, according to any technique known in the art, such as with radiolabels, fluorescent labels, enzymatic labels, proteins, haptens, antibodies, sequence tags, etc.

In performing the methods of the present invention, the oligonucleotides or the target polynucleotide may be either in solution or affixed to a solid support. Generally, allele-specific oligonucleotides will be attached to a solid support, though in certain embodiments of the present invention allele-specific oligonucleotides may be in solution. In some such embodiments, the target polynucleotide is preferably bound to a solid support. In those embodiments where the allele-specific oligonucleotides or the target polynucleotides are attached to a solid support, attachment may be either covalent or non-covalent. Attachment may be mediated, for example, by antibody-antigen-type interactions, poly-L-Lys, streptavidin or avidin-biotin, salt-bridges, hydrophobic interactions, chemical linkages, UV cross-linking, baking, etc. In addition, allele-specific oligonucleotides may be synthesized directly on a solid support or attached to the solid support subsequent to synthesis. In a preferred embodiment, allele-specific oligonucleotides are affixed a solid support such that a free 3′-OH is available for polymerase-mediated primer extension.

Preferably, oligonucleotides are between 10 and 35 nucleotides in length. Most preferably, oligonucleotides are 15 to 30 nucleotides in length. The exact length of a particular oligonucleotide, however, will depend on many factors, which in turn depend on the ultimate function or use of the oligonucleotide. Short primer molecules generally require lower temperatures to form sufficiently stable hybrid complexes with the template. Oligonucleotides, such as primer oligonucleotides are preferably single stranded, but may alternatively be double stranded. If double stranded, the oligonucleotide is generally first treated to separate its strands before being used for hybridization purposes or being used to prepare extension products. Preferably, the oligonucleotide is an oligodeoxyribonucleotide. Oligonucleotides may be synthesized chemically by any suitable means known in the art or derived from a biological sample, as for example, by restriction digestion.

Oligonucleotides may be labeled, according to any technique known in the art, such as with radiolabels, fluorescent labels, bioluminescent label, chemiluminescent label, enzymatic labels, proteins, haptens, antibodies, sequence tags, etc. Methods for labeling are well-known to one of skill in the art.

Probes

Included in the invention are oligonucleotide probes for detection of α_{2C}AR variant polynucleotides and α_{2C}AR polymorphic sites. Probes can be designed to hybridize to e.g., SEQ ID NOS. 1-30. Alternatively, these probes may incorporate other regions of the relevant genomic locus, including intergenic sequences.

Information on the identity of genotypes and haplotypes for the gene of any particular individual as well as the frequency of such genotypes and haplotypes in any particular population of individuals is expected to be useful for a variety of basic research and clinical applications. Thus, the invention also provides compositions for detecting the novel α_{2C}AR polymorphisms and haplotypes identified herein; e.g., genotyping oligonucleotides.

Genotyping oligonucleotides of the invention must be capable of specifically hybridizing to a target region of an α_{2C}AR polynucleotide, i.e., an α_{2C}AR sequence with one or more polymorphisms.

Preferred genotyping oligonucleotides of the invention are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a polymorphic site while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR Protocols, A Guide to Methods and Applications, Academic Press, 1990 and Ruano et al., 87 Proc. Natl. Acad. Sci. USA 6296-6300, 1990. Typically, an allele-specific oligonucleotide will be perfectly complementary to one allele while containing a single mismatch for another allele.

Allele-specific oligonucleotide probes which usually provide good discrimination between different alleles are those in which a central position of the oligonucleotide probe aligns with the polymorphic site in the target region. An allele-specific oligonucleotide primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. Allele-specific oligonucleotide primers hybridizing to either the coding or non-coding strand are contemplated by the invention.

The invention further provides such an allele-specific oligonucleotide, wherein the oligonucleotide is complementary to the target polynucleotide at a region comprising or being nucleotides at position, 59, 222, 281, 358, 569, 574, 712, 946, 1125, 1376, 1673, 1698, 1705, 1942, 2397, 2408, an insertion or deletion from 3601-3612, an insertion or deletion from 4110-4130, 4123, 4394, 3570, 3633, and 3804, of the α_{2C}AR molecule. In one embodiment, a probe for detecting an α_{2C}AR polymorphic site comprises a sequence as disclosed in Table 2, e.g., SEQ ID NOS:52-97.

Other genotyping oligonucleotides of the invention hybridize to a target region located one to several nucleotides downstream of one of the novel polymorphic sites identified herein. Such oligonucleotides are useful in polymerase-mediated primer extension methods for detecting α_{2C}AR gene polymorphisms and thus are referred to herein as “primer-extension oligonucleotides.” In a preferred embodiment, the 3′-terminus of a primer-extension oligonucleotide is a deoxynucleotide complementary to the nucleotide located immediately adjacent to the polymorphic site.

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

Primers

The invention further provides a primer oligonucleotide for amplifying a region of a target polynucleotide, the region comprising a polymorphic site of an α2C-adrenergic receptor molecule (especially one comprising nucleotides at position, 59, 222, 281, 358, 569, 574, 712, 946, 1125, 1376, 1673, 1698, 1705, 1942, 2397, 2408, an insertion or deletion from 3601-3612 an insertion or deletion from 4110-4130, 4123, 4394, 3570, 3633, and 3804 of the α2C-adrenergic receptor molecule), wherein the primer oligonucleotide is substantially complementary to the target polynucleotide, thereby permitting the amplification of the region of the target polynucleotide.

The invention further provides oligonucleotides that can be used to amplify across a single nucleotide polymorphic site of the present invention. The invention further provides oligonucleotides that may be used to sequence the amplified sequence. The invention further provides a method of analyzing a nucleic acid from a DNA sample using amplification and sequencing primers to assess whether a sample contains the reference or variant base (allele) at the polymorphic site, comprising the steps of amplifying a sequence using appropriate primers for amplifying a segment comprising a polymorphic site, sequencing the resulting amplified product using appropriate sequencing primers to sequence the product, and determining whether the variant or reference base or sequence is present at the polymorphic site.

Equivalent primers corresponding to unique sequences occurring 5′ and 3′ to these α_{2C}AR polymorphic sites will be apparent to one of skill in the art. Reasonable equivalent primers include those which hybridize within about 1 kb of the designated primer, and which further are anywhere from about 17 bp to about 27 bp in length. Designing appropriate primer is well known to one of skill in the art. A general guideline for designing primers for amplification of unique human chromosomal genomic sequences is that they possess a melting temperature of at least about 50° C., wherein an approximate melting temperature can be estimated using the Wallace Rule formula Tₘₑₗₜ=[2×(# of A+T)+4×(# of G+C)]. This equation was developed for short DNA oligonucleotides of 14-20 base pairs hybridizing to membrane-bound DNA targets in 0.9 M NaCl. If both target and probe are free in solution, the melt temperature decreases by approximately 7-8° C. as compared to the Wallace Rule value. Optimal design of such primer sequences can be achieved, for example, by the use of commercially available primer selection programs such as Primer 2.1, Primer 3 or GeneFisher and the Genome Database (GDB) project at the URL http://www.gdb.org).

Polypeptides and Antibodies

The invention further provides an isolated α_{2C}AR variant polypeptide having an amino acid sequence encoded by an α_{2C}AR variant polynucleotide described herein.

The present invention also provides antibodies directed against the polypeptides of the present invention. Preferably, such antibodies are capable of discriminating against the reference or variant allele of the polypeptide, preferably at one or more polymorphic sites of the polynucleotide.

Methods of Genotyping

The invention further provides methods of genotyping an individual comprising analyzing a nucleic acid from an individual and determining which nucleotides(s) are present at the α_{2C}AR polymorphic sites. In some embodiments, the nucleotide identity at more than one polymorphic site is determined individually. In other embodiments, the nucleotide identity at more than one polymorphic site is determined simultaneously in one reaction. This type of analysis can be performed on a single individual, or on a plurality of individuals. In some embodiments, the genotyping method is performed on a plurality of individuals who have been tested for the presence or absence of a condition or disease, e.g. heart failure, cardiac arrhythmias, hypertension, behavioral and learning disorders, psychiatric diseases such as depression, obesity, and diabetes mellitus. The presence or absence of disease phenotype or propensity for developing or not developing a disease state can then be correlated with genotype. Alternatively, if a correlation has already been made, the genotyping can be used in methods of prognosis, diagnosis, and treatment selection for a condition or disease.

The invention further relates to using the α_{2C}AR genotyping method to construct haplotypes.

The invention further provides a method of analyzing a nucleic acid from DNA sample(s) from various ethnic populations to identify the nucleotide at an α_{2C}AR polymorphic site in an effort to identify populations at risk of developing diseases including heart failure, cardiac arrhythmias, hypertension, behavioral learning disorders, psychiatric diseases such as depression, obesity, and diabetes mellitus.

This genotyping prediction method is useful for predicting the individual's predisposition to a disease or condition associated with one of the alternative alleles in the α_{2C}AR gene. However, the present invention is not limited to the α_{2C}AR polymorphism or haplotype associations presently known but is applicable to future discoveries of associations between polymorphisms or haplotype and disease, severity of disease, staging of disease, or any other phenotype. It is also contemplated that the above genotyping and haplotyping methods of the invention may be performed in combination with identifying the genotype(s) and/or haplotype(s) for other genomic regions. The above described genotyping methods are useful in methods for determining the frequency of an α_{2C}AR genotype or haplotype in a population. The method comprises determining the genotype or the haplotype pair for the α_{2C}AR gene that is present in each member of the population and calculating the frequency at which any particular α_{2C}AR genotype or haplotype is found in the population. The population may be a reference population, a family population, a same sex population, a population group, a trait population (e.g., a group of individuals exhibiting a trait of interest such as a medical condition or response to a therapeutic treatment).

Frequency data for such α_{2C}AR genotypes or haplotypes in reference and trait populations are useful for identifying an association between a trait and any novel α_{2C}AR polymorphism, genotype or haplotype. The trait may be any detectable phenotype, including but not limited to susceptibility to a disease or response to a treatment. The method involves obtaining data on the frequency of the genotype(s) or haplotype(s) of interest in a reference population as well as in a population exhibiting the trait. Frequency data for one or both of the reference and trait populations may be obtained by genotyping or haplotyping the α_{2C}AR gene in each individual in the populations using one of the methods described above. The haplotypes for the trait population may be determined directly or, alternatively, by the predictive genotype to haplotype approach described above. In another embodiment, the frequency data for the reference and/or trait populations is obtained by accessing previously determined frequency data, which may be in written or electronic form. For example, the frequency data may be present in a database that is accessible by a computer. Once the frequency data is obtained, the frequencies of the genotype(s) or haplotype(s) of interest in the reference and trait populations are compared. In a preferred embodiment, the frequencies of all genotypes and/or haplotypes observed in the populations are compared. If a particular genotype or haplotype for the α_{2C}AR gene is more frequent in the trait population than in the reference population at a statistically significant amount, then the trait is predicted to be associated with that α_{2C}AR genotype or haplotype. Preferably, the α_{2C}AR genotype or haplotype being compared in the trait and reference populations is selected from the full-genotypes and full-haplotypes shown in Tables 3 and 4 respectively, or from sub-genotypes and sub-haplotypes derived from these genotypes and haplotypes.

Determination of Polymorphism Identity

Many methods are available for determination of polymorphism identity, e.g., identifying the nucleotide at an α_{2C}AR polymorphic site in a sample. These methods are well known to one of skill in the art, and one of skill will know that detecting a specific polymorphic allele will depend, in part, upon the molecular nature of the polymorphism. Techniques include: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Oligonucleotides necessary for amplification may be selected, for example, from within the α2C gene loci, either flanking the marker of interest (as required for PCR amplification) or directly overlapping the marker (as in ASO hybridization).

A preferred detection method is allele specific hybridization using probes overlapping a region of at least one allele of an α_{2C}AR haplotype and having about 5, 10, 20, 25, or 30 nucleotides around the α_{2C}AR polymorphic site. In one embodiment of the invention, several probes capable of hybridizing specifically to multiple α_{2C}AR polymorphic sites are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

Various techniques and technologies may be used for synthesizing dense arrays of biological materials on or in a substrate or support. For example, Affymetrix GeneChip arrays are synthesized in accordance with techniques sometimes referred to as VLSIPS (Very Large Scale Immobilized Polymer Synthesis) technologies. Some aspects of VLSIPS and other microarray and polymer (including protein) array manufacturing methods and techniques have been described in U.S. patent Ser. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Applications Nos. PCT/US99/00730 (International Publication No. WO 99/36760) and PCT/US01/04285, which are all incorporated herein by reference in their entireties for all purposes.

Patents that describe synthesis techniques in specific embodiments include U.S. Pat. Nos. 6,486,287, 6,147,205, 6,262,216, 6,310,189, 5,889,165, 5,959,098, and 5,412,087, all hereby incorporated by reference in their entireties for all purposes. Nucleic acid arrays are described in many of the above patents, but the same techniques generally may be applied to polypeptide arrays.

An allele comprising an α_{2C}AR haplotype may also be detected indirectly, e.g., by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications.

The identity of polymorphisms may also be determined using a mismatch detection technique, including but not limited to the RNase protection method using riboprobes (Winter et al., Proc. Natl. Acad. Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253 (1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp 321-340, 1996) or denaturing gradient gel electrophoresis (DGGE) (Wartell et al., Nucl. Acids Res. 18:2699-2706, 1990; Sheffield et al., Proc. Natl. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524. Related methods are disclosed in WO91/02087, WO90/09455, WO95/17676, and U.S. Pat. Nos. 5,302,509 and 5,945,283. Extended primers containing a polymorphism may be detected by mass spectrometry as described in U.S. Pat. No. 5,605,798. Another primer extension method is allele-specific PCR (Ruano et al., Nucl. Acids Res. 17:8392, 1989; Ruano et al., Nucl. Acids Res. 19, 6877-6882, 1991; WO 93/22456; Turki et al., J. Clin. Invest. 95:16352C641, 1995). In addition, multiple polymorphic sites may be investigated by simultaneously amplifying multiple regions of the nucleic acid using sets of allele-specific primers as described in Wallace et al. (WO89/10414).

A variety of methods are available for detecting the presence of a particular single nucleotide polymorphic (SNP) allele in an individual including using a specialized exonuclease-resistant nucleotide, as disclosed, e.g., in Mundy, C. R. (U.S. Pat. No. 4,656,127); a solution-based method (Cohen, D. et al. (PCT Appln. No. WO91/02087)) where a primer is employed that is complementary to allelic sequences immediately 3′ to a polymorphic site and uses a labeled dideoxynucleotide derivatives; dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. Other techniques include generation of small signal molecules by invasive cleavage followed by mass spectrometry or immobilized padlock probes and rolling-circle amplification; primer-guided nucleotide incorporation procedures for assaying polymorphic sites in DNA (Komher, J. S. et al., Nucl. Acids. Res. 17:7779-7784 (1989); Sokolov, B. P., Nucl. Acids Res. 18:3671 (1990); Syvanen, A-C., et al., Genomics 8:684-692 (1990); Kuppuswamy, M. N. et al., Proc. Natl. Acad. Sci. (U.S.A.) 88:1 1432C147 (1991); Prezant, T. R. et al., Hum. Mutat. 1:1592C64 (1992); Ugozzoli, L. et al., GATA 9:1072C12 (1992); Nyren, P. et al., Anal. Biochem. 208 1712C75 (1993)), the protein truncation test (PTT) (Roest, et. al., (1993) Hum. Mol. Genet. 2:1719-21; van der Luijt et. al., (1994) Genomics 20:14).

In addition to methods that focus primarily on the detection of one nucleic acid sequence, profiles may also be assessed in such detection schemes. Fingerprint profiles may be generated, for example, by utilizing a differential display procedure, Northern analysis and/or RT-PCR.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:18742C878), transcriptional amplification system, (Kwoh, D. Y. et al., 1989, Proc. Natl. Acad. Sci. USA 86:11732C177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like. PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection methods allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of an α_{2C}AR haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of an α_{2C}AR haplotype is identified by alterations in restriction enzyme cleavage patterns. For example, sample and control DNA is isolated, amplified (optionally using appropriate primers from Table 2), digested with one or more restriction endonucleases, and fragment length sizes are determined by gel electrophoresis.

In yet another embodiment, any of a variety of sequencing reactions known in the art can be used to directly sequence the allele. It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylanine or osmium tetroxide and with piperidine) can be used to detect mismatched bases in RNA/RNA or RNA/DNA or DNA/DNA heteroduplexes (Myers, et al. (1985) Science 230:1242). See, for example, Cotton et al (1988) Proc. Natl. Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:16572C662). According to an exemplary embodiment, a probe based on an allele of an α2C locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify an α2C locus allele. For example, single strand conformation polymorphism (SSCP) may be used to detect differences in electrophoretic mobility between mutant and wild type nucleic acids (Orita et al. (1989) Proc. Natl. Acad. Sci USA 86:2766, see also Cotton (1993) Mutat Res 285:1252C44; and Hayashi (1992) Genet Anal Tech Appl 9:73-79). In another embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

In another embodiment, identification of the allelic variant is carried out using an oligonucleotide ligation assay (OLA), as described, e.g., in U.S. Pat. No. 4,998,617 and in Landegren, U. et al. ((1988) Science 241:10772C080). A nucleic acid detection assay that combines attributes of PCR and OLA can also be used (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of an α2C locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well.

Any cell type or tissue may be utilized to obtain nucleic acid samples for use in the diagnostics described herein. In a preferred embodiment, the DNA sample is obtained from a bodily fluid, e.g., blood, obtained by known techniques (e.g., venipuncture) or saliva. Alternatively, nucleic acid tests can be performed on dry samples (e.g., hair or skin). When using RNA or protein, the cells or tissues that may be utilized must express an α2C gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

Kits

The invention also provides a kit for performing the assays described herein, e.g., suitable for genetic testing. Such a kit includes a means for identifying the nucleotide at least one α_{2C}AR polymorphic site in a subject. In one embodiment, the means of nucleotide identification is primers for amplifying one or more regions of α2C-adrenergic receptor nucleic acid encompassing regions where at least one of the polymorphisms is found; and allele-specific oligonucleotides, specific for both mutant and wild-type alleles of at least one of these polymorphic site sequences. The kit can also contain control target polynucleotides. The kit can also contain additional reagents and components including, e.g., nucleic acid amplification reagents, polymerase, nucleic acid amplification reagents, restrictive enzymes, buffers, a nucleic acid sampling device, DNA purification device, deoxynucleotides, etc.

Particularly preferred primers for use in the diagnostic method of the invention include SEQ ID NOS. 32-97 as shown in Tables 1 and 2.

A “control” in the kit may be a positive or negative control and can be in the form of patient nucleic acid samples, cloned target polynucleotides, plasmids or bacterial strains carrying positive and negative control DNA. Further, the control sample may contain the positive (or negative) products of the allele detection technique employed. For example, where the allele detection technique is PCR amplification, followed by size fractionation, the control sample may comprise DNA fragments of the appropriate size. Likewise, where the allele detection technique involves detection of a mutated protein, the control sample may comprise a sample of mutated protein. However, it is preferred that the control sample comprises the material to be tested. For example, the controls may be a sample of genomic DNA or a cloned portion of the α2C gene cluster. Preferably, however, the control sample is a highly purified sample of genomic DNA where the sample to be tested is genomic DNA.

The oligonucleotides present in the kit may be used for amplification of the region of interest or for direct allele specific oligonucleotide (ASO) hybridization to the α_{2C}AR polymorphic site.

As described herein, suitable primers for the detection of α_{2C}AR polymorphic site can be readily designed using sequence information and standard techniques known in the art for the design and optimization of primers sequences.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include nucleic acid sampling means. Nucleic acid sampling means are well known to one of skill in the art and can include, but not be limited to substrates, such as filter papers, the AmpliCard (University of Sheffield, Sheffield, England S10 2JF; Tarlow, J W, et al., J. of Invest. Dermatol. 103:387-389 (1994)) and the like; DNA purification reagents such as Nucleon kits, lysis buffers, proteinase solutions and the like; PCR reagents, such as 10× reaction buffers, thermostable polymerase, dNTPs, and the like; and allele detection means such as the HinfI restriction enzyme, allele specific oligonucleotides, degenerate oligonucleotide primers for nested PCR from dried blood.

Methods of Diagnosing and Treatment Selection

Information obtained using the assays and kits described herein (alone or in conjunction with information on another genetic defect or environmental factor, which contributes to the disease or condition that is associated with an α_{2C}AR haplotype) is useful for determining whether a non-symptomatic subject has or is likely to develop the particular disease or condition. In addition, the information can allow a more customized approach to preventing the onset or progression of the disease or condition. For example, this information can enable a clinician to more effectively prescribe a therapy that will address the molecular basis of the disease or condition.

In yet a further aspect, the invention features methods for treating or preventing the development of a disease or condition that is associated with an α_{2C}AR haplotype in a subject by administering to the subject an appropriate therapeutic of the invention. In still another aspect, the invention provides in vitro or in vivo assays for screening test compounds to identify therapeutics for treating or preventing the development of a disease or condition that is associated with an α_{2C}AR haplotype.

In yet another embodiment, the invention provides a method for identifying an association between an α_{2C}AR haplotype and a trait. In preferred embodiments, the trait is susceptibility to a disease, disease severity, the staging of a disease or response to a drug. Such methods have applicability in developing diagnostic tests and therapeutic treatments for one or more conditions selected from the group consisting of congestive heart failure, arrhythmia, ischemic heart disease, hypertension, migraine, anaphylaxis, obesity, diabetes, myasthenia gravis (MG), depression, anxiety, psychosis, attention deficit disorder and premature labor. In other preferred embodiments, the drug is an agonist or antagonist of α_{2C}AR.

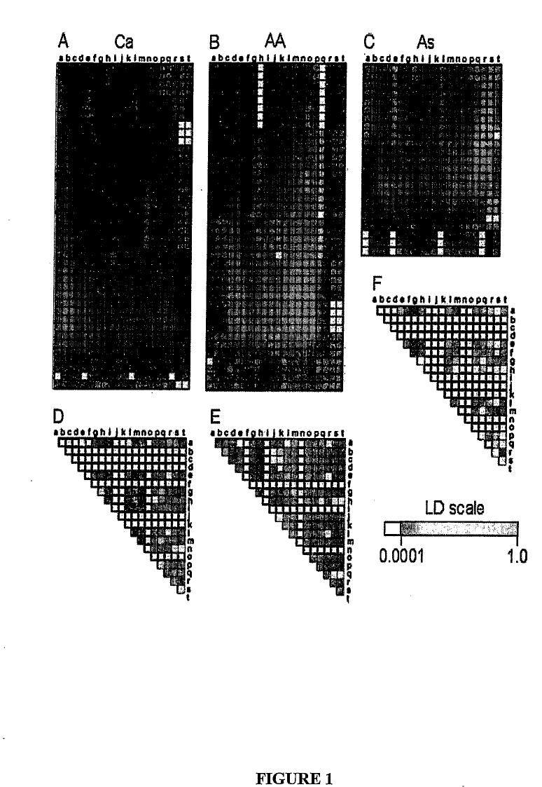

The α_{2C}AR haplotypes are associated with the development, progression, and treatment response of certain diseases or conditions. Therefore, for example, detection of the alleles comprising a haplotype, alone or in conjunction with another means in a subject can indicate that the subject has or is predisposed to the development of a particular disease or condition. However, because these alleles are in linkage disequilibrium with other alleles, the detection of such other linked alleles can also indicate that the subject has or is predisposed to the development of a particular disease or condition. For example, it is known that the nucleotide at position 712 is always a “G” if the nucleotide at position 1705 is a “C” and the nucleotide at position 712 is always an “A” if the nucleotide at position 1705 is an “A.” Due to varying degrees of linkage-disequilibrium between polymorphic sites (see FIG. 1D, 1E, 1F), this type of relationship may allow prediction of traits between other polymorphic sites.

The statistical correlation between a pathological disorder and an α_{2C}AR polymorphism does not necessarily indicate that the polymorphism directly causes the disorder. Rather the correlated polymorphism may be a benign allelic variant that is linked to (i.e., in linkage disequilibrium with) a disorder-causing mutation that has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci that are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype.

Thus, the determination of an individual's likelihood for developing a particular disease of condition can be made by characterizing one or more disease-associated polymorphic alleles (or even one or more disease-associated haplotypes) without necessarily determining or characterizing the causative genetic variation.

On the other hand, the disease or drug-response polymorphism or mutation can be within the gene being investigated. In fact, disease predisposition or drug-response may require several genetic variants in the gene (the haplotype), and thus the unique combination of these variants can augment a test's predictive power.

Correlations Between Treatment and α_{2C}AR Genotype

The present invention further relates to a method of predicting the response of an individual having a particular haplotype or combination of one or more polymorphisms listed in Table 3 and/or 4 to a particular pharmaceutical agent or other administered therapeutic. The method comprises the steps of (a) determining which base or combination of bases is present in an individual at any one or more of the polymorphic sites shown in Tables 3 and/or 4 as described above, and (b) administering a pharmaceutical agent or other therapeutic agent that is anticipated to have the most advantageous therapeutic effect.

In order to deduce a correlation between clinical response to a treatment and a α_{2C}AR genotype or haplotype, it is necessary to obtain data on the clinical responses exhibited by a population of individuals who received the treatment, hereinafter the “clinical population.” This clinical data may be obtained by analyzing the results of a clinical trial that has already been run and/or the clinical data may be obtained by designing and carrying out one or more new clinical trials. As used herein, the term “clinical trial” means any research study designed to collect clinical data on responses to a particular treatment, and includes but is not limited to phase I, phase II and phase III clinical trials. Standard methods are used to define the patient population and to enroll subjects.

It is preferred that selection of individuals for the clinical population comprises grading such candidate individuals for the existence of the medical condition of interest and then including or excluding individuals based upon the results of this assessment. This is important in cases where the symptom(s) being presented by the patients can be caused by more than one underlying condition, and where treatment of the underlying conditions are not the same. For example, patients with congestive heart failure may have a different prognosis and treatment plan depending on whether they have high or low levels of norepinephrine. The α_{2C}AR controls norepinephrine release, so by knowing which haplotype of the α_{2C}AR a patient has, one could then divide patients into those with haplotypes that cause high norepinephrine vs. those that cause low norepinephrine. In other words, patients with similar or the same presentation of disease could be distinguished by haplotype, and prognosis and treatment could be optimized for the patient based on this distinguishing feature.

The therapeutic treatment of interest, or the control treatment (active agent or placebo in controlled trials), is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the α_{2C}AR gene for each individual in the trial population is genotyped and/or haplotyped, which may be done before or after administering the treatment.

After both the clinical and polymorphism data have been obtained, correlations between individual response and α_{2C}AR genotype or haplotype content are created. Correlations may be produced in several ways. In one method, individuals are grouped by their α_{2C}AR genotype or haplotype (or haplotype pair) (also referred to as a polymorphism group), and then the averages and standard deviations of continuous clinical responses exhibited by the members of each polymorphism group are calculated.

These results are then analyzed to determine if any observed variation in clinical response between polymorphism groups is statistically significant. Statistical analysis methods which may be used are described in L. D. Fisher and G. vanBelle, “Biostatistics: A Methodology for the Health Sciences”, Wiley-Interscience (New York) 1993. This analysis may also include a regression calculation of which polymorphic sites in the α_{2C}AR gene give the most significant contribution to the differences in phenotype.

A second method for finding correlations between α_{2C}AR haplotype content and clinical responses uses predictive models based on error-minimizing optimization algorithms. One of many possible optimization algorithms is a genetic algorithm (R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (Press et al., “Numerical Recipes in C: The Art of Scientific Computing”, Cambridge University Press (Cambridge) 1992, Ch. 10), neural networks (E. Rich and K. Knight, “Artificial Intelligence”, 2nd Edition McGraw-Hill; New York, 1991, Ch. 18), standard gradient descent methods (Press et al., supra Ch. 10), or other global or local optimization approaches (see discussion in Judson, supra) could also be used.

Correlations may also be analyzed using analysis of variation (ANOVA) techniques to determine how much of the variation in the clinical data is explained by different subsets of the polymorphic sites in the α_{2C}AR gene. ANOVA is used to test hypotheses about whether a response variable is caused by or correlated with one or more traits or variables (in this case, polymorphism groups) that can be measured (Fisher and vanBelle, supra, Ch. 10). These traits or variables are called the independent variables. To carry out ANOVA, the independent variable(s) are measured and individuals are placed into groups based on their values for these variables. In this case, the independent variable(s) refers to the combination of polymorphisms present at a subset of the polymorphic sites, and thus, each group contains those individuals with a given genotype or haplotype pair. The variation in response within the groups and also the variation between groups are then measured. If the within-group response variation is large (people in a group have a wide range of responses) and the response variation between groups is small (the average responses for all groups are about the same) then it can be concluded that the independent variables used for the grouping are not causing or correlated with the response variable. For instance, if people are grouped by month of birth (which should have nothing to do with their response to a drug) the ANOVA calculation should show a low level of significance. However, if the response variation is larger between groups than within groups, the F-ratio (=“between groups” divided by “within groups”) is greater than one. Large values of the F-ratio indicate that the independent variable is causing or correlated with the response. The calculated F-ratio is preferably compared with the Critical F-distribution value at whatever level of significance is of interest. If the F-ratio is greater than the Critical F-distribution value, then one may be confident that the individual's genotype or haplotype pair for this particular subset of polymorphic sites in the α_{2C}AR gene is at least partially responsible for, or is at least strongly correlated with the clinical response.

From the analyses described above, a mathematical model may be readily constructed by the skilled artisan that predicts clinical response as a function of α_{2C}AR genotype or haplotype content. Preferably, the model is validated in one or more follow-up clinical trials designed to test the model.

The identification of an association between a clinical response and a genotype or haplotype (or haplotype pair) for the α_{2C}AR gene may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic sites in the α_{2C}AR gene), a serological test, or a physical exam measurement. The only requirement is that there be a good correlation between the diagnostic test results and the underlying α_{2C}AR genotype or haplotype that is in turn correlated with the clinical response. In a preferred embodiment, this diagnostic method uses the predictive haplotyping method described above.

### Pharmacogenomics

Knowledge of the particular alleles associated with a susceptibility to developing a particular disease or condition, alone or in conjunction with information on other genetic defects contributing to the particular disease or condition allows a customization of the prevention or treatment in accordance with the individual's genetic profile, the goal of “pharmacogenomics.” Thus, comparison of an individual's α_{2C}AR profile to the population profile for a cardiac disorder permits the selection or design of drugs such as α_{2C}AR agonists or antagonists, beta-blockers or other therapeutic regimens that are expected to be safe and efficacious for a particular patient or patient population (i.e., a group of patients having the same genetic alteration).

In another pharmacogenomic application, the α_{2C}AR polymorphisms or haplotypes influence the effect of a drug acting at another target that is involved with the α_{2C}AR in the pathologic pathways of a disease.

Another aspect of the invention is a method for predicting an individual's response to a beta-blocker, which comprises assigning an α_{2C}AR haplotype pair to the individual and using the assigned haplotype pair to make a response prediction

In one embodiment, the assigning step comprises determining a genotype of an individual's α_{2C}AR gene and using the genotype to assign the haplotype pair. Based on Table 4, one skilled in the art would recognize that an individual could be heterozygous or homozygous for any given haplotype, and any combination of haplotypes could be possible. For example, an individual could have one chromosome with haplotype 1, and one chromosome having haplotype 2. Likewise, any given haplotype could be observed in combination with the same or any other haplotype.

In addition, the ability to target populations expected to show the highest clinical benefit, based on genetic profile can enable: 1) the repositioning of already marketed drugs; 2) the rescue of drug candidates whose clinical development has been discontinued as a result of safety or efficacy limitations, which are patient subgroup-specific; and 3) an accelerated and less costly development for candidate therapeutics and more optimal drug labeling (e.g., since measuring the effect of various doses of an agent as a function of genotype or haplotype is useful for optimizing effective dose).

The treatment of an individual with a particular therapeutic can be monitored by determining protein, mRNA and/or transcriptional level. Depending on the level detected, the therapeutic regimen can then be maintained or adjusted (increased or decreased in dose). In a preferred embodiment, the effectiveness of treating a subject with an agent comprises the steps of: (i) obtaining a preadministration sample from a subject prior to administration of the agent; (ii) detecting the level of expression or activity of a protein, mRNA or genomic DNA in the preadministration sample; (iii) obtaining one or more post-administration samples from the subject; (iv) detecting the level of expression or activity of the protein, mRNA or genomic DNA in the post-administration sample; (v) comparing the level of expression or activity of the protein, mRNA or genomic DNA in the preadministration sample with the corresponding protein, mRNA or genomic DNA in the postadministration sample, respectively; and (vi) altering the administration of the agent to the subject accordingly.

Cells of a subject may also be obtained before and after administration of a therapeutic to detect the level of expression of genes other than an α2C gene to verify that the therapeutic does not increase or decrease the expression of genes which could be deleterious. This can be done, e.g., by using the method of transcriptional profiling. Thus, mRNA from cells exposed in vivo to a therapeutic and mRNA from the same type of cells that were not exposed to the therapeutic could be reverse transcribed and hybridized to a chip containing DNA from numerous genes, to thereby compare the expression of genes in cells treated and not treated with the therapeutic.

Gene Therapy

Polynucleotides comprising an α_{2C}AR variant or fragment may be useful for therapeutic purposes. For example, where a patient could benefit from expression, decreased expression or increased expression, of a particular α_{2C}AR haplotype, an expression vector encoding the haplotype may be administered to the patient. The patient may be one who lacks the α_{2C}AR haplotype or may already have at least one copy of that haplotype.

In further embodiments, the invention provides for treatment of heart failure by detecting and remediating (e.g., via conventional gene therapy techniques) the α_{2C}AR genomic polymorphism either systemically or in the affected tissues, as well as diseases such as cardiac arrhythmias, hypertension, behavioral and learning disorders, psychiatric diseases such as depression, obesity, and diabetes mellitus. Alternatively, such treatment may be attained through detection of the polymorphism or variant protein, and by application of appropriate medications, e.g., for blocking a post-synaptic adrenergic receptor.

In addition to heart failure, the invention can be applied to the diagnosis, prediction and/or treatment of ventricular dysfunction and other cardiac diseases. The foregoing techniques can likewise be applied to other mammals in a manner similar to that of humans.

Therapeutics

The invention also comprises screening for therapeutics. Therapeutic for diseases or conditions associated with an α2C polymorphism or haplotype refers to any agent or therapeutic regimen (including pharmaceuticals, nutraceuticals and surgical means) that prevents or postpones the development of or alleviates the symptoms of the particular disease or condition in the subject. The therapeutic can be a polypeptide, peptidomimetics, nucleic acid or other inorganic or organic molecule, preferably a “small molecule” including vitamins, minerals and other nutrients.

A small molecule as used herein, is meant to refer to a composition, which has a molecular weight of less than about 5 kD and most preferably less than about 4 kD. Small molecules can be nucleic acids, peptides, peptidomimetics, carbohydrates, lipids or other organic to or inorganic molecules.

The therapeutic can be an agonist or an antagonist. An agonist can be a wild-type molecule or protein or derivative thereof having at least one bioactivity of the wild-type, e.g., receptor binding activity. An agonist can also be a compound that upregulates expression of a gene or which increases at least one bioactivity of a protein. An agonist can also be a compound that increases the interaction of a polypeptide with another molecule, e.g., a receptor.

An antagonist can be a compound that inhibits or decreases the interaction between a protein and another molecule, e.g., a receptor or an agent that blocks signal transduction or post-translation processing. Accordingly, a preferred antagonist is a compound that inhibits or decreases binding to a receptor and thereby blocks subsequent activation of the receptor. An antagonist can also be a compound that downregulates expression of a gene or which reduces the amount of a protein present. The antagonist can be a dominant negative form of a polypeptide, e.g., a form of a polypeptide that is capable of interacting with a target peptide, e.g., a receptor, but which does not promote the activation of the receptor. The antagonist can also be a nucleic acid encoding a dominant negative form of a polypeptide, an antisense nucleic acid, or a ribozyme capable of interacting specifically with an RNA. Yet other antagonists are molecules that bind to a polypeptide and inhibit its action. Such molecules include peptides, e.g., forms of target peptides which do not have biological activity, and which inhibit binding to receptors.

For treatment, therapeutics are generally formulated as a pharmaceutical composition. Pharmaceutical compositions usually comprise a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

Whether it is a polypeptide, antibody, nucleic acid, small molecule or other pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of the disorder or disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

A composition can be administered alone or in combination with other treatments, either simultaneously or sequentially dependent upon the condition to be treated.

Therapeutics Screening

Based on the identification of α_{2C}AR polymorphic sites and haplotypes that cause or contribute to the development of an α_{2C}AR condition, the invention further features cell-based or cell free assays for screening test compounds and identifying therapeutics. In one embodiment, a cell expressing an α_{2C}AR, or a receptor for a protein that is encoded by a gene which is in linkage disequilibrium with an α2C gene, on the outer surface of its cellular membrane is incubated in the presence of a test compound alone or in the presence of a test compound and another protein and the interaction between the test compound and the receptor or between the protein (preferably a tagged protein) and the receptor is detected, e.g., by using a microphysiometer (McConnell et al. (1992) Science 257:1906).

Cellular or cell-free assays can also be used to identify compounds which modulate expression of an α2C haplotype or a gene in linkage disequilibrium therewith, modulate translation of an mRNA, or which modulate the stability of an mRNA or protein. Accordingly, in one embodiment, a cell which is capable of producing an α_{2C}AR is incubated with a test compound and the amount of α_{2C}AR produced is measured and compared to that produced from a cell which has not been contacted with the test compound. The specificity of the compound vis a vis the protein can be confirmed by various control analysis, e.g., measuring the expression of one or more control genes. In particular, this assay can be used to determine the efficacy of antisense, ribozyme and triplex compounds.

Cell-free assays can also be used to identify compounds that are capable of interacting with a protein, to thereby modify the activity of the protein. Such a compound can, e.g., modify the structure of a receptor thereby affecting its ability to bind to a ligand. In a preferred embodiment, cell-free assays for identifying such compounds consist essentially of a reaction mixture containing a protein and a test compound or a library of test compounds in the presence or absence of a binding partner. A test compound can be, e.g., a derivative of a binding partner, e.g., a biologically inactive target peptide, or a small molecule.

Accordingly, one exemplary screening assay of the present invention includes the steps of contacting an α_{2C}AR protein or functional fragment thereof with a test compound or library of test compounds and detecting the formation of complexes. For detection purposes, the molecule can be labeled with a specific marker and the test compound or library of test compounds labeled with a different marker. Interaction of a test compound with a protein or fragment thereof can then be detected by determining the level of the two labels after an incubation step and a washing step. The presence of two labels after the washing step is indicative of an interaction. An interaction between molecules can also be identified by using real-time BIA (Biomolecular Interaction Analysis, Pharmacia Biosensor AB) which detects surface plasmon resonance (SPR), an optical phenomenon. This technique is further described, e.g., in BIAtechnology Handbook by Pharmacia.

Another exemplary screening assay of the present invention includes the steps of (a) forming a reaction mixture including: (i) an α_{2C}AR, (ii) an appropriate ligand, and (iii) a test compound; and (b) detecting interaction of the ligand and receptor. A statistically significant change (potentiation or inhibition) in the interaction of the ligand and receptor in the presence of the test compound, relative to the interaction in the absence of the test compound, indicates a potentially active test compound. The compounds of this assay can be contacted simultaneously. Alternatively, a receptor can first be contacted with a test compound for an appropriate amount of time, following which the ligand is added to the reaction mixture. The efficacy of the compound can be assessed by generating dose response curves from data obtained using various concentrations of the test compound. Moreover, a control assay can also be performed to provide a baseline for comparison.

Complex formation between a ligand and receptor may be detected by a variety of techniques. Modulation of the formation of complexes can be quantitated using, for example, detectably labeled proteins such as radiolabeled, fluorescently labeled, or enzymatically labeled proteins or receptors, by immunoassay, or by chromatographic detection.

Typically, it will be desirable to immobilize either the ligand or the receptor to facilitate separation of complexes from uncomplexed forms of one or both of the proteins, as well as to accommodate automation of the assay. Binding of ligand and receptor can be accomplished in any vessel suitable for containing the reactants. Examples include microtitre plates, test tubes, and micro-centrifuge tubes: In one embodiment, a fusion protein can be provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the receptor, e.g., an ³⁵S-labeled receptor, and the test compound, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes can be dissociated from the matrix, separated by SDS-PAGE, and the level of ligand or receptor found in the bead fraction quantitated from the gel using standard electrophoretic techniques such as described in the appended examples. Other techniques for immobilizing proteins on matrices are also available for use in the subject assay. For instance, either ligand or receptor can be immobilized utilizing conjugation of biotin and streptavidin.

Transgenic Animals

The present invention also provides genetically modified animals comprising one or more of the novel α_{2C}AR genomic polymorphic variants described herein and methods for producing such animals. Such animals are useful for studying expression of the α_{2C}AR haplotypes in vivo, for in vivo screening and testing of drugs targeted against α_{2C}AR protein, and for testing the efficacy of therapeutic agents and compounds targeting the α_{2C}AR in a biological system.

Transgenic animals can also be made to identify agonists and antagonists or to confirm the safety and efficacy of a candidate therapeutic. Transgenic animals of the invention can include non-human animals containing an α_{2C}AR genomic polymorphic variant described herein under the control of an appropriate endogenous promoter or under the control of a heterologous promoter.

Transgenic animals are made by transgenic techniques well known in the art. The nucleic acid is introduced into the cell, directly or indirectly by introduction into a precursor of the cell, by way of deliberate genetic manipulation; such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. Moreover, “transgenic animal” also includes those recombinant animals in which gene disruption of one or more genes is caused by human intervention, including both recombination and antisense techniques. The term is intended to include all progeny generations. Thus, the founder animal and all F1, F2, F3, and so on, progeny thereof are included.

Computer Systems

The present invention also provides a computer system for storing and displaying polymorphism data determined for the α_{2C}AR gene. The computer system comprises a computer processing unit; a display; and a database containing the polymorphism data. The polymorphism data includes the polymorphisms, the genotypes and the haplotypes identified for the α_{2C}AR gene in one or both of the reference population and the patient population. In a preferred embodiment, the computer system is capable of producing a display showing α_{2C}AR haplotypes organized according to their evolutionary relationships.

The present invention also provides for a computer readable medium having stored thereon a sequence selected from the group consisting of a nucleic acid code comprising a contiguous span of at least 12 nucleotides of any of SEQ ID NOS. 1 to 31 or the complements thereof. The present invention also provides for a computer system comprising a processor and a data storage device wherein the data storage device has stored thereon a sequence selected from the group consisting of a nucleic acid code comprising a contiguous span of at least 12 nucleotides of any of SEQ ID NOS. 1 to 31 or the complements thereof. In one embodiment, the computer system further comprises a sequence comparer and a data storage device having reference sequences stored thereon.

Any or all analytical and mathematical operations involved in practicing the methods of the present invention may be implemented by a computer. In addition, the computer may execute a program that generates views (or screens) displayed on a display device and with which the user can interact to view and analyze large amounts of information relating to the α_{2C}AR gene and its genomic variation, including chromosome location, gene structure, and gene family, gene expression data, polymorphism data, genetic sequence data, and clinical data population data (e.g., data on ethnogeographic origin, clinical responses, genotypes, and haplotypes for one or more populations). The α_{2C}AR polymorphism data described herein may be stored as part of a relational database (e.g., an instance of an Oracle database or a set of ASCII flat files). These polymorphism data maybe stored on the computer's hard drive or may, for example, be stored on a CD ROM or on one or more other storage devices accessible by the computer. For example, the data may be stored on one or more databases in communication with the computer via a network.

As will be appreciated by one of skill in the art, the present invention may be embodied as a method, data processing system or program products. Accordingly, the present invention may take the form of data analysis systems, methods, analysis software, and so on. Software written according to the present invention typically is to be stored in some form of computer readable medium, such as memory, or CD-ROM, or transmitted over a network, and executed by a processor. For a description of basic computer systems and computer networks, see, e.g., Introduction to Computing Systems: From Bits and Gates to C and Beyond by Yale N. Paft, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both of which are hereby incorporated by reference for all purposes.

In one embodiment, the present invention provides for a system for providing correlations between one or more probe-set identifiers and one or more biological sequences, comprising: an input manager constructed and arranged to receive a user selection of the probe-set identifiers; a correlator constructed and arranged to associate the probe-set identifiers with probe sequences and to correlate the probe sequences with the one or more biological sequences as described herein; and an output manager constructed and arranged to enable for display a graphical user interface including one or more graphical elements representing one or more of the probe sequences associated with the probe-set identifiers and one or more graphical elements representing the one or more biological sequences. In an alternate embodiment, the one or more biological sequences include any one or more of sequences of EST, DNA, cDNA, RNA, cRNA, mRNA, gene region, gene, gene transcripts, gene transcription products, chromosome, peptide, protein fragment, protein, or residue counting reference.

Optionally, the probe-set identifiers or probe sequences are associated with probes of a probe array constructed and arranged to detect or measure any one or any combination of biological, biochemical or genetic metrics including gene expression, genotype, SNP, haplotype, or targets including antibodies, cell membrane receptors, monoclonal antibodies and antisera reactive with specific antigenic determinants, drugs, oligonucleotides, nucleic acids, peptides, proteins, cofactors, lectins, sugars, polysaccharides, cells, cellular membranes, or organelles.

In another embodiment, the method further comprises one or more of the steps of: receiving a user selection of the probe-set identifiers; the user selection is provided, optionally at least in part, using the Internet; the user selection is provided, at least in part, using a graphical user interface; the graphical user interface includes elements for receiving a user selection including a list of probe-set identifiers in a computer-readable file; the graphical user interface includes one or more user input elements for receiving a user selection of one or more query biological sequences, or identifiers of one or more query biological sequences; and the method further includes the act of associating the one or more query biological sequences, or their identifiers, with one or more probe-set identifiers.

Biallelic Arrays

The present invention also provides for the use of a polynucleotide for use in determining the identity of nucleotides at a map-related biallelic marker selected from the group consisting of the biallelic markers of SEQ ID NOS. 1 to 31. It also provides for where the determining is performed in a hybridization assay, sequencing assay, microsequencing assay, or an enzyme-based mismatch detection assay. The present invention also provides for the use of a polynucleotide for use in amplifying a segment of nucleotides comprising a map-related biallelic marker selected from the group consisting of the biallelic markers of SEQ ID NOS. 1 to 31. The present invention also provides for the use where the polynucleotide is attached to a solid support, e.g., an array. Preferably, the array is addressable.

The present invention also provides for the use of a computer readable medium having stored thereon the sequence of a polynucleotide comprising a contiguous span of 12 nucleotides selected from the group consisting of SEQ ID NOS. 1 to 31 comprising a map-related biallelic marker, to analyze a nucleotide sequence. The present invention also provides for the use of a computer system comprising a processor and a data storage device wherein the data storage device has stored thereon the sequence of a polynucleotide comprising a contiguous span of 12 nucleotides selected from the group consisting of SEQ ID NOS. 1 to 31 comprising a map-related biallelic marker, to analyze a nucleotide sequence. The present invention also provides for the use of a computer system, wherein the computer system further comprises a sequence comparer and a data storage device having reference sequences stored thereon.

Linkage Disequilibrium

Linkage disequilibrium refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at greater than expected frequencies are then said to be in “linkage disequilibrium.” The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern.

In addition to the allelic patterns described above, as described herein, one of skill in the art can readily identify other alleles (including polymorphisms and mutations) that are in linkage disequilibrium with an allele associated with a disease or disorder. For example, a nucleic acid sample from a first group of subjects without a particular disorder can be collected, as well as DNA from a second group of subjects with the disorder. The nucleic acid sample can then be compared to identify those alleles that are over-represented in the second group as compared with the first group, wherein such alleles are presumably associated with a disorder, which is caused or contributed to by inappropriate α_{2C}AR function and or regulation. Alternatively, alleles that are in linkage disequilibrium with an allele that is associated with the disorder can be identified, for example, by genotyping a large population and performing statistical analysis to determine which alleles appear more commonly together than expected. Preferably the group is chosen to be comprised of genetically related individuals. Genetically related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms that are linked along a chromosome in a founder population to redistribute through genetic cross-over events. Thus race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-causing mutation is a meta-stable state. Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

Methods

The reference sequence for the intronless human α_{2C}AR gene is SEQ ID NO:1 GenBank accession number AY605898. This reference sequence is based on the most common sequence of the α_{2C}AR gene that we found in our population (Haplotype 1, see below). For purposes of maintaining consistency, nucleic acid residues in this report are numbered 1-4625, and are from the numbering in AY605898. Unless otherwise noted, all nucleotide positions are in reference to AY605898 (the GenBank accession number).

We note that within this sequence the A of the initiation of translation codon ATG is at position 2638. So as to provide additional insight as to 5′ upstream vs. other regions, in table 3 we also define nucleotide positions based on the translation start site. Thus the 5′ promoter is −2638 to −906, the 5′UTR is −905 to −1, the coding is +1 to +1390 and the 3′UTR is +1391 to +1929.

Polymorphism Discovery. Direct sequencing of PCR fragments spanning the α_{2C}AR gene amplified from genomic DNA from an index repository consisting of 30 ethnically diverse individuals was performed using an ABI Prism 3700 Sequencer (Applied Biosystems). Primer sets and reaction conditions used for polymorphism discovery are shown in Table 1. Buffer designations are to those provided in Invitrogen's PCR Optimizer Kit (Cat. No. K1220-01) and AccuPrime Taq DNA Polymerase Hi Fidelity Kit (Cat. No. 12346-086) documentation materials and are as follows. DMSO, when included, is included at a final concentration of 10%. Each letter-designated buffer appearing in Table 1 and Table 2 includes 60 mM Tris-Hcl and 15 mM ammonium sulfate. In addition, buffer A includes 1.5 mM MgCl₂, pH 8.5; buffer B includes 2 mM MgCl₂, pH 8.5; buffer C includes 2.5 mM MgCl₂, pH 8.5; buffer D includes 3.5 mM MgCl₂, pH 8.5; buffer F includes 2 mM MgCl₂, pH 9.0; buffer G includes 2.5 mM MgCl₂, pH 9.0; buffer H includes 3.5 mM MgCl₂, pH 9.0; buffer I includes 1.5 mM MgCl₂, pH 9.5; buffer J includes 2 mM MgCl₂, pH 9.5. HiFi buffer is 60 mM Tris-SO₄, 18 mM ammonium sulfate, and included MgSO₄ at the indicated final concentrations, pH 8.9. The cited pH values are for 5× letter-designated buffers and for 10× HiFi buffer. These values were not adjusted following dilution of buffer stocks to 1× strength. For some fragments, direct sequencing yielded equivocal results, so PCR fragments were cloned into the vector pCR2.1-TOPO and multiple colonies from transformed bacteria were expanded. Isolated DNA from at least 5 independent clones was subsequently sequenced. Electronic data from sequencing was compared to the reference sequence using MacVector 7.2 (Accelrys), and variants were confirmed by direct inspection of the electropherograms.

Primers used for amplification of α_{2C}AR fragments 1 through 10 and described in Table 1 are also identified as SEQ ID NOS: 32-51, and included in the Sequence Listing.

TABLE 1

Primers and reaction conditions used
for α_{2C}AR polymorphism discovery

| Region | | | | Anneal | Size |
|---|---|---|---|---|---|
| Amplified | Frag. | Primer Sets | Buffer | T (° C.) | (bp) |
| Promoter | 1 | 5′-tcccactggaggaaggacagcctac-3′ | C | 65 | 516 |
| | | 5′-cacgttggagccaaagccttctgt-3′ | | | |
| | 2 | 5′-tttagtaaggatggtgaccc-3′ | C | 65 | 577 |
| | | 5′-ttctcccaaacgtccagaaacgaa-3′ | | | |
| | 3 | 5′-cagccgctcttcatgatgacctcc-3′ | C | 65 | 607 |
| | | 5′-gctgctggagcatgaatcataact-3′ | | | |
| | 4 | 5′-acttcaatcactgctaacatgggt-3′ | D + | 59 | 529 |
| | | 5′-gagcccgcggccgcctggtcgaac-3′ | DMSO | | |
| 5′UTR | 5 | 5′-cttagaaagagcagcttctggaactc-3′ | HiFi/ | 65 | 764 |
| | | 5′-ctgccgagcgtgtaagtgcagagc-3′ | 2.5 mM | | |
| | | | MgSO₄ + | | |
| | | | DMSO | | |
| | 6 | 5′-ggaaagtaaagttggagacggagg-3′ | HiFi/ | 65 | 743 |
| | | 5′-acggtgaagacgatgaggaagccca-3′ | 10 mM | | |
| | | | MgSO₄ + | | |
| | | | DMSO | | |
| Coding | 7 | 5′-tagctcgcgggaggaccatggcgtccc-3′ | HiFi/ | 65 | 665 |
| | | 5′-gatgcaggaggacaggatgtaccaggtct-3′ | 10 mM | | |
| | | | MgSO₄ + | | |
| | | | DMSO | | |
| | 8 | 5′-ccatcgtcgccgtgtggctcatct-3′ | HiFi/ | 65 | 723 |
| | | 5′-aggcctcgcggcagatgccgtaca-3′ | 7.5 mM | | |
| | | | MgSO₄ + | | |
| | | | DMSO | | |
| | 9 | 5′-gagaagcgcttcacctttgtgc-3′ | F + | 54 | 393 |
| | | 5′-gccctggaggccaatccatcc-3′ | DMSO | | |
| 3′UTR | 10 | 5′-ccgacggaggagaaggggcttcag-3′ | C | 65 | 630 |
| | | 5′-aactcgaacccccgacccccataga-3′ | | | |

Genotype and Haplotype Determination. Unphased genotypes were determined using an expanded index repository of 105 anonymous genomic DNA samples (Coriell Institute) representing 40 Caucasians, 40 African-Americans, and 25 Asians. For the identified polymorphisms, the presence of the variant allele in a PCR product resulted in gain or loss of a restriction enzyme site, thereby providing a rapid genotyping method. See Table 2 for assay conditions. In instances of repeated homozygosity, the chromosomal organization of some polymorphisms was evident by inspection. In cases of multiply heterozygous samples, haplotypes were determined by a molecular approach. This was carried out by PCR amplification of the gene (4,625 bp) using the Elongase Amplification System (Invitrogen) followed by subcloning of the product and subsequent genotyping of single clones, yielding phased genotypes. We also compared the composition of haplotypes delineated by this molecular approach with haplotypes imputed from unphased genotype data by an algorithmic method (14), using the computer program PHASE (University of Washington).

Primer pairs used for α_{2C}AR genotyping and described in Table 2 are also described herein as SEQ ID NOS:52-97 and included in the Sequence Listing.

TABLE 2

| Primer pairs, reaction conditions, and restriction enzymes used | | | | | |
|---|---|---|---|---|---|
| for α_{2C}AR polymorphism genotyping. | | | | | |
| | | | | | Rxn |
| Location | | | Anneal | Size | Enzyme |
| Code | Primer Pairs | Buffer | T (° C.) | (bp) | Change |
| A | 5′-ggcctcccccttcctcataacacctcctGa-3′* | B | 65 | 270 | Lose |
| | 5′-acacacctgcttccttcatggtcg-3′ | | | | Tfi I |
| | | | | | site |
| B | 5′-tcccactggaggaaggacagccatc-3′ | C | 65 | 510 | Gain |
| | 5′-cacgttggagccaaagccttctgt-3′ | | | | Kas I |
| | | | | | site |
| C | 5′-tcccactggaggaaggacagccatc-3′ | C | 65 | 510 | Gain |
| | 5′-cacgttggagccaaagccttctgt-3′ | | | | Bsp HI |
| | | | | | site |
| D | 5′-tcccactggaggaaggacagccatc-3′ | C | 65 | 510 | Gain |
| | 5′-cacgttggagccaaagccttctgt-3′ | | | | Hinc |
| | | | | | II |
| | | | | | site |
| E | 5′-ttagtaaggatggtgaccc-3′ | C | 61 | 251 | Gain |
| | 5′-cttcgttcttcaccatgtttg-3′ | | | | BstNI |
| | | | | | site |
| f | 5′-tgaaatatttgctttctgccgggccccggAt-3′* | C + | 65 | 200 | Lose |
| | 5′-tctgagctcctgaagggcgcca-3′ | DMSO | | | Mbo I |
| | | | | | site |
| g | 5′-aggcccatcatctgaaatatttgc-3′ | A | 60 | 216 | Lose |
| | 5′-tctgagctcctgaagggcgcca-3′ | | | | Dde I |
| | | | | | site |
| h | 5′-cagccgctcttcatgatgacctcc-3′ | C | 65 | 607 | Lose |
| | 5′-gctgctggagcatgaatcataact3′ | | | | Mae II |
| | | | | | site |
| i | 5′-cagccgctcttcatgatgacctcc-3′ | C | 65 | 607 | Gain |
| | 5′-gctgctggagcatgaatcataact-3′ | | | | Pvu II |
| | | | | | site |
| j | 5′-acttcaatcactgctaacatgggt-3′ | C + | 59 | 258 | Lose |
| | 5′-tgatggcagagcaacctctcgca-3′ | DMSO | | | Aci I |
| | | | | | site |
| k | 5′-acttcaatcactgctaacatgggt-3′ | F + | 59 | 529 | Lose |
| | 5′-gagcccgcggccgcctggtcgaac-3′ | DMSO | | | Dde I |
| | | | | | site |
| l | 5′-agtcttaaaagagcagcttctggaactctcc-3′ | B + | 57 | 178 | Lose |
| | 5′-gagcccgcggccgcctggtcgaac-3′ | DMSO | | | Eci I |
| | | | | | site |
| m | 5′-acttcaatcactgctaacatgggt-3′ | F + | 59 | 529 | Lose |
| | 5′-gagcccgcggccgcctggtcgaa-3′ | DMSO | | | Ngo |
| | | | | | MIV |
| n | 5′-cttagaaagagcagcttctggaactc-3′ | H + | 65 | 764 | Lose |
| | 3′-ctgccgagcgtgtaagtgcagagc-3′ | DMSO | | | Not I |
| | | | | | site |
| o | 5′-gagccctagccggccggatgggag-3′ | N + | 65 | 339 | Gain |
| | 5′-cccagcagccggccctcggccgt-3′ | DMSO | | | Ava I |
| | | | | | site |
| p | 5′-gtcgccgctcgctccgggcgcct-3′ | G + | 65 | 272 | Gain |
| | 5′-agcgccggggacgccatggtcct-3′ | DMSO | | | Bsr BI |
| | | | | | site |
| q | 5′-agccggacgagagcagcgca-3′ | F + | 65 | 384/ | Lose |
| | 5′-aggcctcgcggcagatgccgtaca-3′ | DMSO | | 372 | Nci I |
| | | | | | site |
| r | 5′-ccgacggaggagaaggggcttcag-3′ | C + | 65 | 163/ | Size |
| | 5′-gccctggaggccaatccatcc-3′ | DMSO | | 142 | |
| s | 5′-gagaagcgcttcacctttgtgc-3′ | F + | 54 | 395 | Gain |
| | 5′-gccctggaggccaatcc-3′ | DMSO | | | Sac I |
| | | | | | site |
| t | 5′-gggcaggagcttggcagagagatagccg-3′ | C + | 65 | 251 | Lose |
| | 5′-ccttggtcagacggggatggggagtggtagAgt-3′* | DMSO | | | Hinf I |
| | | | | | site |
| u | 5′-agccggacgagagcagcgca-3′ | F + | 65 | 384/ | Gain |
| | 5′-aggcctcgcggcagatgccgtaca-3′ | DMSO | | 372 | Sfo I |
| | | | | | site |
| v | 5′-agccggacgagagcagcgca-3′ | F + | 65 | 384/ | Gain |
| | 5′-aggcctcgcggcagatgccgtaca-3′ | DMSO | | 372 | Bme1580I |
| | | | | | site |
| w | 5′-cgcttcacctttgtgctggctgtggtcatgCgc-3′* | B | 65 | 179 | Lose |
| | 5′-gtgtagatgaccgggttgagca-3′ | | | | Hha I |
| | | | | | site |

*sequence of primer was altered to engineer a restriction enzyme site change in the presence of the polymorphism. Altered nucleotide is in CAPS.

Constructs and Cell Transfections. For expression studies, PCR products of the 4,625 bp α_{2C}AR gene derived from human genomic DNA were cloned into the vector pCR2.1-TOPO. This vector lacks a eukaryotic-responsive promoter and thus expression of these constructs is directed by the included α_{2C}AR promoter sequence. Each construct was sequenced to verify the haplotype and to insure that it differed from other constructs only at the appropriate polymorphic sites. The human neuroblastoma cell line BE(2)-C was utilized as the host cell for transient transfections and were grown in monolayers as described (15). Transient transfections with Lipofectamine 2000 (Invitrogen) were performed using 12 ug of each haplotype construct as described by the manufacturer. Two days post-transfection, cells were harvested for radioligand binding or real time RT-PCR.

Radioligand Binding. Confluent monolayers of BE(2)-C cells were washed three times with phosphate-buffered saline, lysed in a hypotonic buffer (5 mM Tris, 2 mM EDTA, pH 7.4), detached by scraping with a rubber policeman, and centrifuged at 42,000×g for 10 min. Crude membranes were resuspended in buffer (75 mM Tris, 12.5 mM MgCl₂, 2 mM EDTA, pH 7.4) and saturation binding assays were performed in triplicate as described (16) using [³H]yohimbine and 10 uM phentolamine to define nonspecific binding.

Reverse Transcription and Real-time PCR. Total RNA was prepared using Trizol reagent (Invitrogen). Reverse transcription of total RNA was performed after Dnase I digestion (Ambion) using MuLV reverse transcriptase (Applied Biosystems), 500 ng total RNA, and 2.5 uM random hexamers. Identical incubations were also performed in the absence of reverse transcriptase. Forward and reverse primers (5′-tttgcacctcgtcgatcgt and 5′-cctgcgtcaccgaccagta) for quantitative real time PCR amplified a 66 bp fragment from an invariant portion of the α_{2C}AR coding region. PCR reactions consisted of a 50-fold dilution of the reverse transcriptase reaction (˜10 ng), 300 nM each forward and reverse primers, and 25 ul Sybr Green Master Mix (Applied Biosystems). Cycle conditions were 50° C. for 2 min, 95° C. for 10 min, and 40 cycles of 95° C. for 15 sec and 60° C. for 1 min. Each sample was assayed in triplicate. Samples derived from reactions carried out in the absence of reverse transcriptase were tested simultaneously with experimental samples and consistently yielded no amplification. For each amplification, a standard curve was generated using 0.1-100 ng cDNA prepared from stock BE(2)-C total RNA to determine the relative levels of α_{2C}AR present in each sample. Reactions containing primers corresponding to endogenous GAPDH (5′-atggaaatcccatcaccatctt and 5′-cgccccacttgattttgg) were also performed to control for small variations in the amount of template in each reaction.

Miscellaneous. Agreement between genotypes and those predicted by Hardy-Weinberg equilibrium were assessed by Chi-square tests with one degree of freedom. Linkage disequilibrium was calculated as Δ (17). Haplotype-tagged polymorphisms were determined using the computer program SNP-tagger (18). Comparisons of results from radioligand binding and real-time RT-PCR were analyzed by repeated measures ANOVA and post-hoc t-tests. Significance was considered when P<0.05 after correction for multiple comparisons by the Newman-Keuls method. Data are reported as mean±SE.

Identification of Multiple Polymorphic Sites in the α_{SC}AR Gene

Polymorphisms of the α_{2C}AR gene were ascertained from a total of 105 individuals composed of three ethnic groups. The interrogated sequence of this intronless gene consisted of ˜4,625 contiguous bases. In doing so, multiple sequence variants were identified with allele frequencies of >1% compared to the reference sequence in at least one of the ethnic groups. The allele frequencies, locations, and identifier codes for the polymorphisms are shown in Table 3. Of the 23 variants, 21 were SNPs, and two were deletion polymorphisms. Within the α_{2C}AR coding region, we identified only one nonsynonymous variant (position q, Table 3) which is the previously described 12 bp in frame deletion of nucleotides 3601-3612 that results in loss of amino acids 322-325 (12). We also noted three synonymous SNPs (i.e., SNPs that do not alter the encoded amino acid) in the open reading frame at nucleotide positions 3570, 3633, and 3804. While these do not alter the encoded protein, we are cognizant that they may be in linkage-disequilibrium with other SNPs that are functional, and as such could be used as genetic markers for functional SNPs. They are thus included in the tables and haplotype analysis, but not in the functional studies. In the non-coding regions of the α_{2C}AR gene, thirteen sequence variants were found within the α_{2C}AR promoter region, three within the 5′UTR, and three within the 3′UTR. In the 3′UTR, a 21 bp deletion of nucleotides 4110-4130 (location r) was identified 94 bp downstream of the α_{2C}AR translational stop site. Interestingly, another 3′UTR SNP at position 4123 (location s) occurs within this deletion region, thus in the presence of the r polymorphism, the s variant cannot be present on the same chromosome. The distribution of homozygous and heterozygous alleles in each ethnic group was not different from that predicted from Hardy-Weinberg equilibrium (P>0.05).

TABLE 3
Polymorphic sites in α_{2C}AR

| | | Nucleotide | | | | | |
|---|---|---|---|---|---|---|---|
| | | relative to | | | | | |
| | | GenBank | | | Minor Allele | | |
| | Location | AY605898 | Nucleotide | | Frequency | | |
| Region | code | (SEQ ID NO: 1) | relative to ATG start site | Alleles | Ca | AA | As |
| Promoter | a | 59 | −2579 | T/C | 8.8 | 6.3 | 34.0 |
| Promoter | b | 222 | −2416 | C/G | 0 | 10.0 | 0 |
| Promoter | c | 281 | −2357 | C/T | 0 | 10.0 | 0 |
| Promoter | d | 358 | −2280 | G/T | 0 | 5.0 | 0 |
| Promoter | e | 569 | −2069 | C/T | 7.5 | 2.5 | 34.0 |
| Promoter | f | 574 | −2064 | C/T | 0 | 0 | 2.0 |
| Promoter | g | 712 | −1926 | G/A | 1.3 | 1.3 | 2.0 |
| Promoter | h | 946 | −1692 | T/G | 6.3 | 38.8 | 10.0 |
| Promoter | i | 1125 | −1513 | T/G | 2.5 | 0 | 0 |
| Promoter | j | 1376 | −1262 | C/A | 0 | 7.5 | 0 |
| Promoter | k | 1673 | −965 | G/C | 0 | 11.3 | 0 |
| Promoter | l | 1698 | −940 | G/A | 7.5 | 2.5 | 34.0 |
| Promoter | m | 1705 | −933 | C/A | 1.3 | 1.3 | 2.0 |
| 5′UTR | n | 1942 | −696 | C/G | 11.3 | 0 | 0 |
| 5′UTR | o | 2397 | −241 | C/G | 0 | 7.5 | 0 |
| 5′UTR | p | 2408 | −230 | T/C | 5.0 | 2.5 | 8.0 |
| Coding- | q | 3601 to 3612 | +964 to +975 | Ins/Del | 6.3 | 42.5 | 14.0 |
| nonsyn | | | | | | | |
| 3′UTR | r | 4110 to 4130 | +1483 to +1503 | Ins/Del | 8.8 | 5.0 | 36.0 |
| 3′UTR | s | 4123 | +1486 | T/C* | 34.2 | 71.1 | 37.5 |
| 3′UTR | t | 4394 | +1757 | G/C | 31.3 | 67.5 | 20.8 |
| Coding-syn | u | 3570 | +933 | G/C | 6.3 | 42.5 | 10.0 |
| Coding-syn | v | 3633 | +996 | G/A | 23.8 | 15.0 | 14.0 |
| Coding-syn | w | 3804 | +1167 | C/T | 6.3 | 41.3 | 10.0 |

Ca, Caucasians;
AA, African-American;
As, Asian;
syn, synonymous;
nonsysn, nonsynonymous.
*SNP at s is located within the insertion sequence site r only, so in the deletion form of r, the s genotype is not applicable.

Examination of the frequencies of each α_{2C}AR polymorphism revealed that some were cosmopolitan (present in all ethnic groups) while others displayed a high degree of population specificity (Table 3). Fourteen polymorphisms (˜61%) were present in all ethnic groups and represented variation in all regions of the α_{2C}AR gene. For many cosmopolitan polymorphisms, there were nevertheless substantial differences in the allele frequencies between ethnic groups. In African-Americans, polymorphisms at positions h, q, s, t, u and v were present at significantly higher allele frequencies compared to Caucasians and Asians (P<0.001). As discussed below, these four variants also show a high degree of linkage disequilibrium within this population. In Asians, polymorphisms at positions a, e, l, and r are significantly more common in this group as compared to Caucasians and African-Americans (P<0.0001). When examining population-specific variants, African-Americans had the highest number (six), with two present in Caucasians, and a single population-specific variant observed for Asians. Overall, the frequencies of polymorphisms (both cosmopolitan and population-specific) in each group ranged from 0 to as high as 70%. Indeed, SNP locations s and t in African-Americans are very common, in which case the minor alleles in the total population are actually the major alleles in African-Americans. The diversity of the α_{2C}AR gene is greater than that reported from re-sequencing DNA from a multiethnic population at 313 genes randomly selected from the genome (19). This is particularly so for the promoter, which has a prevalence of 7.6 SNPs/kb for the α_{2C}AR compared to an average of 2.6 SNPs/kb for promoters in the genome-wide study. Interestingly, the coding region of the α_{2C}AR has only one non-synonymous polymorphism (representing 0.7/kb), which is lower than expected for an intronless G-protein coupled receptor (20). Thus the majority of the genetic plasticity of the α_{2C}AR has evolved due to variations in the non-coding regions.

FIG. 1A-C shows a graphical display of α_{2C}AR genotypes for each DNA sample grouped by population. Blue and yellow indicate homozygotes for the reference and variant alleles, respectively, and red indicates heterozygotes. A color version of FIG. 1 is published in Small, Mialet-Perez, et al., “Polymorphisms of cardiac presynaptic α_{2c} adrenergic receptors: Diverse intragenic variability with haplotype-specific functional effects,” PNAS 101 (35): 13020-13025 (Aug. 31, 2004) and also is available at http://www.pnas.org/cgi/content/full/101/35/13020. Individual DNA samples are clustered to show sites with similar genotype patterns. Genotype data was used to calculate the degree of linkage disequilibrium (A) between all pairs of polymorphic loci (FIG. 1 D-F). Of the 23 variants, relatively few pairs exhibit high levels of linkage disequilibrium (defined as Δ value ≧0.8). As shown in FIG. 1, among all ethnic groups linkage disequilibrium was observed for a:e:l:r:g:m, h:p:q:v:w, and s:t. At sites a:e:l:r and h:p:q:v:w, the degree of linkage disequilibrium observed in Caucasians and Asians was high between all possible pairs. However, in African-Americans, the Δ values for these pairs were significantly lower. For example, Δ was 0.89 for h:p in Caucasians, but was 0.20 for African-Americans. Unique combinations of polymorphisms showing strong linkage disequilibrium only in African-Americans were also noted and included the paired combinations of b, c, j, and k. When considering the Del322-325 polymorphism (q), the SNP at location h is in strong linkage disequilibrium with this polymorphism in all ethnic groups. Interestingly, in Caucasians and Asians only, the SNP at location p also showed high linkage disequilibrium with Del322-325.

α_{2C}AR Polymorphisms are Organized into 30 Haplotypes.

These 23 variable loci of the α_{2C}AR gene were found to be organized into a total of 30 haplotypes within the entire cohort as determined by the molecular method (Table 4). The raw unphased genotypes from individuals with polymorphism with frequencies ≧5% in any one population were also utilized to infer haplotypes by the algorithmic method, and we found complete concordance between the two approaches. Of note, GenBank accession numbers U72648, NM_{—}000683 and AC141928 contain sequence reported as human α_{2C}AR with flanking sequence, however these do not contain any of the haplotypes of Table 3, including haplotype 1 which is the most common in an ethnically mixed population. Thus we did not find any individual that has the U72648, NM_{—}000683 or AC141928 sequence.

The α_{2C}AR haplotypes described in Table 4 are also described herein as SEQ ID NOS:1-30 and included in the Sequence Listing.

TABLE 4

| Haplotypes of the alpha_{2C} AR gene in 105 individuals. | | | | | | | | | | | | | | | | | | | | | | | | | | |
|---|---|---|---|---|---|---|---|---|---|---|---|---|---|---|---|---|---|---|---|---|---|---|---|---|---|---|
| loc | | | | | | | | | | | | | | | | | | | | | | | | | | |
| code | 59 | 222 | 281 | 358 | 569 | 574 | 712 | 946 | 1125 | 1376 | 1673 | 1698 | 1705 | 1942 | 2397 | 2408 | 3601 to 3612 | 4110 to 4130 | 4123 | 4394 | 3570 | 3633 | 3804 | | | |
| HAP# | a | b | c | d | e | f | g | h | I | j | k | l | m | n | o | p | q | r | s | t | u | v | w | AA | Ca | As |
| 1 | T | C | C | G | C | C | G | T | T | C | G | G | C | C | C | T | Ins | Ins | T | G | G | G | C | 21.3 | 58.8 | 36.0 |
| 2 | T | C | C | G | C | C | G | T | T | C | G | G | C | C | C | T | Ins | Ins | C | C | G | A | C | 10.0 | 12.5 | 8.0 |
| 3 | T | C | C | G | C | C | G | G | T | C | G | G | C | C | C | T | Del | Ins | C | C | C | G | T | 21.3 | 0 | 10.0 |
| 4 | T | C | C | G | C | C | G | G | T | C | G | G | C | C | G | T | Del | Ins | C | C | C | G | T | 6.25 | 0 | 0 |
| 5 | T | G | T | G | C | C | G | T | T | A | C | G | C | C | C | T | Ins | Ins | C | C | G | G | C | 7.5 | 0 | 0 |
| 6 | T | C | C | G | C | C | G | T | T | C | G | G | C | C | C | T | Del | Ins | C | C | C | G | T | 3.75 | 0 | 0 |
| 7 | T | C | C | T | C | C | G | G | T | C | G | G | C | C | C | T | Del | Ins | C | C | C | G | T | 5 | 0 | 0 |
| 8 | C | C | C | G | T | C | G | T | T | C | G | A | C | C | C | T | Ins | Del | * | G | G | G | C | 2.5 | 7.5 | 22.0 |
| 9 | T | C | C | G | C | C | G | G | T | C | G | G | C | C | C | C | Del | Ins | C | C | C | G | T | 2.5 | 3.75 | 0 |
| 10 | T | C | C | G | C | C | G | T | T | C | G | G | C | C | C | T | Ins | Del | * | G | G | G | C | 1.25 | 0 | 2.0 |
| 11 | T | C | C | G | C | C | G | G | T | C | G | G | C | C | C | T | Ins | Ins | T | G | G | G | C | 2.5 | 0 | 0 |
| 12 | T | G | T | G | C | C | G | T | T | C | C | G | C | C | C | T | Ins | Ins | C | C | G | G | C | 2.5 | 0 | 0 |
| 13 | T | C | C | G | C | C | G | T | G | C | G | G | C | C | C | T | Ins | Ins | T | G | G | G | C | 0 | 2.5 | 0 |
| 14 | T | C | C | G | C | C | G | T | T | C | G | G | C | G | C | T | Ins | Ins | C | C | G | A | C | 0 | 11.3 | 0 |
| 15 | C | C | C | G | C | C | G | G | T | C | G | G | C | C | C | T | Del | Ins | C | C | C | G | T | 1.25 | 0 | 0 |
| 16 | C | C | C | G | C | C | G | T | T | C | G | G | C | C | C | T | Ins | Ins | C | C | G | A | C | 1.25 | 0 | 0 |
| 17 | C | C | C | G | C | C | G | T | T | C | G | G | C | C | C | T | Ins | Del | * | G | G | G | C | 1.25 | 0 | 0 |
| 18 | T | C | C | G | C | C | A | T | T | C | G | G | A | C | C | T | Del | Ins | T | G | G | G | C | 1.25 | 0 | 0 |
| 19 | C | C | C | G | C | C | G | G | T | C | G | G | C | C | C | C | Del | Del | * | C | C | G | T | 0 | 1.25 | 0 |
| 20 | T | C | C | G | C | C | A | G | T | C | G | G | A | C | C | T | Del | Ins | C | C | C | G | T | 0 | 1.25 | 0 |
| 21 | C | C | C | G | T | C | G | T | T | C | G | A | C | C | C | C | Ins | Del | * | G | G | G | C | 0 | 0 | 4.0 |
| 22 | C | C | C | G | T | C | A | T | T | C | G | A | A | C | C | T | Ins | Del | * | G | G | G | C | 0 | 0 | 2.0 |
| 23 | C | C | C | G | T | T | G | T | T | C | G | A | C | C | C | T | Ins | Del | * | G | G | G | C | 0 | 0 | 2.0 |
| 24 | T | C | C | G | C | C | G | T | T | C | G | G | C | C | C | C | Ins | Ins | T | G | G | G | C | 0 | 0 | 2.0 |
| 25 | T | C | C | G | C | C | G | T | T | C | G | G | C | C | C | C | Ins | Ins | C | C | G | G | C | 0 | 0 | 2.0 |
| 1A | T | C | C | G | C | C | G | T | T | C | G | G | C | C | C | T | Ins | Ins | T | G | G | A | C | 2.5 | 0 | 2.0 |
| 2A | T | C | C | G | C | C | G | T | T | C | G | G | C | C | C | T | Ins | Ins | C | C | G | G | C | 3.75 | 1.25 | 4.0 |
| 4A | T | C | C | G | C | C | G | G | T | C | G | G | C | C | G | T | Del | Ins | C | C | C | A | T | 1.25 | 0 | 0 |
| 6A | T | C | C | G | C | C | G | T | T | C | G | G | C | C | C | T | Del | Ins | C | C | C | G | C | 1.25 | 0 | 0 |
| 8A | C | C | C | G | T | C | G | T | T | C | G | A | C | C | C | T | Ins | Del | * | G | G | A | C | 0 | 0 | 4.0 |

Shown are the allele frequencies (in %) of the haplotypes for each ethnic group. See Table 1 for location code and alleles. AA, African-American; Ca, Caucasian; As, Asian; *, not applicable

The haplotypic variation of this gene that we found was substantial, particularly in African-Americans and Asians, where the most common haplotype represents only 21% and 36% of individuals, respectively. As shown in Table 4, there is substantial ethnic variation in α_{2C}AR haplotype frequencies. Only four haplotypes (1, 2, 8 and 2A) are present in all three ethnic groups, and the frequencies at which these haplotypes occur in each group varies considerably. Haplotypes 1 and 2 are the most common haplotypes in all groups and differ in frequency between groups by up to 2-fold. For haplotype 8, there is greater disparity, with allele frequencies of 22% in Asians compared to 2.5% and 7.5% in African-Americans and Caucasians, respectively. In terms of population-specific haplotypes, there are 12 in African-Americans, 4 in Caucasians and 6 in Asians.

We found a complex distribution of the cardiomyopathic α2_{C}Del322-325 polymorphism within the context of these haplotypes. Del322-325 is present primarily in haplotypes 3, 4, 6, and 7 (haplotype frequencies ≧5%) and at lower frequencies in haplotypes 9, 15, 18, 4A and 6A in the African-American group. In Caucasians, Del322-325 is present only in haplotypes 9, 19, and 20, while in Asians it is present exclusively in haplotype 3. Overall, the Del322-325 polymorphism is partitioned into 11 different haplotypes. Approximately 43% of those with the Del322-325 are haplotype 3, while the remainder are distributed amongst several other haplotypes with frequencies varying from 18% to 2% of the Del322-325 population. Thus, heterogeneity within the context of the cardiomyopathic Del322-325 polymorphism is substantial.

Haplotypes Direct Cellular Expression of α_{2C}AR Transcript and Protein.

As noted above, only a single nonsynonymous variant, Del322-325 (location code q) was identified in the coding region. This polymorphism has been recently characterized in stably transfected CHO cells and has been shown to confer reduced coupling of the receptor to multiple signaling pathways (12). We were thus prompted to ascertain the effects of polymorphic variation within the α_{2C}AR gene on receptor expression. For such studies, we felt it was critical to consider polymorphisms within a haplotype context, since it is the net effect from multiple variable sites that potentially direct a phenotype. We thus utilized a strategy whereby the constructs for transfections are devoid of viral or other promoters, but rather consist of the entire 4,625 bp α_{2C}AR sequence encompassing the various haplotypes using this whole-gene transfection method. Expression of the α_{2C}AR is directed/maintained by its own promoter, 5′UTR, and 3′UTR and thus the effects of polymorphisms as they occur in haplotypes within these regions on expression of α_{2C}AR transcripts and protein can thus be ascertained. A neuronal cell line, BE(2)-C, which is of human origin and expresses low levels of α2AR, was utilized as the host cell for transfections. All haplotypes that had allele frequencies of ≧5% in any one of the three ethnic groups were studied (haplotypes 1, 2, 3, 4, 5, 6, 7, 8, and 14).

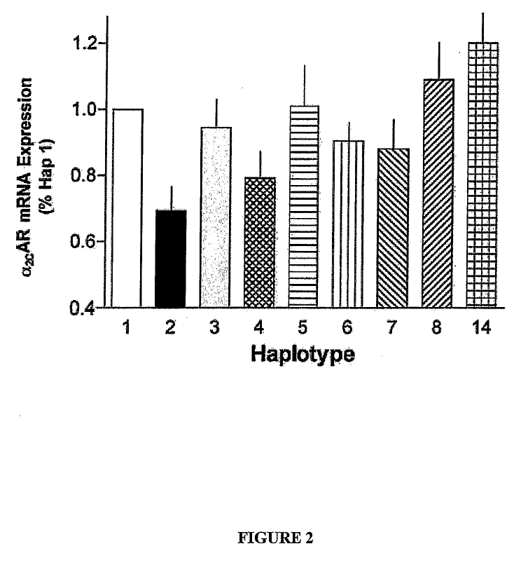

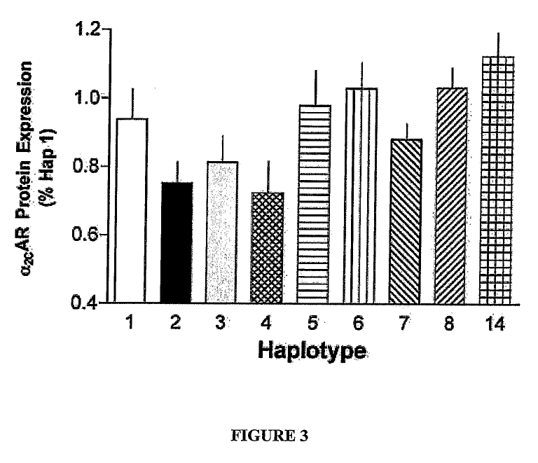

Results from transfection studies where mRNA was quantitated by real-time RT-PCR are shown in FIG. 2. α_{2C}AR mRNA expression was significantly related to haplotype (P<0.001 by ANOVA, n=6 experiments). Haplotype 14 had statistically greater expression than all other haplotypes, which was most apparent compared to low expressing haplotypes 2 and 4 and the intermediate expressing haplotypes 6 and 7 (P<0.001). Haplotypes 2 and 4 had lower expression than all other haplotypes (P values between 0.01 and 0.001). This grouping thus leaves haplotypes 3 and 5 as having essentially wild-type (haplotype 1) expression phenotypes. Haplotype 8 also had relatively higher expression as compared to haplotypes 2, 4 (P<0.001), 7 (P<0.01), and 6 (P<0.05). Given the potential limitations of this transfection approach in representing a haplotype-dependent effect that might be manifested in various cells in an intact organism, expression of α_{2C}AR protein was also considered as a phenotypic endpoint. α_{2C}AR receptor density was determined in the transfected cells by quantitative radioligand binding using the α2AR ligand [³H]yohimbine. There was a significant relationship between α_{2C}AR protein expression and haplotype (P<0.0001 by ANOVA, n=9 experiments, FIG. 3). There was reasonable overall agreement between the haplotype effect on mRNA and protein expression. One notable difference was with haplotype 3, which in the radioligand binding studies had a low level of protein expression that was similar to haplotypes 2 and 4. One might consider, then, that haplotypes 2, 3, and 4 represent a low expression “cluster”.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

1. Liggett, S. B., Shah, S. D. & Cryer, P. E. (1989) Amer. J. Physiol. 256, E600-E609.
2. van Campen, L. C., Visser, F. C. & Visser, C. A. (1998) J. Cardiovasc. Pharmacol. 32 Suppl 1, S31-S35.
3. Israel, E., Drazen, J. M., Liggett, S. B., Boushey, H. A., Cherniack, R. M., Chinchilli, V. M., Cooper, D. M., Fahy, J. V., Fish, J. E., Ford, J. G. et al. (2000) Amer. J. Respir. Crit. Care Med. 162, 75-80.
4. Small, K. M., McGraw, D. W. & Liggett, S. B. (2003) Annu. Rev. Pharmacol. Toxicol. 43, 381-411.
5. Small, K. M., Wagoner, L. E., Levin, A. M., Kardia, S. L. R. & Liggett, S. B. (2002) N. Engl. J. Med. 347, 1135-1142.
6. Gerson, M. C., Wagoner, L. E., McGuire, N. & Liggett, S. B. (2003) J. Nucl. Cardiol. 10, 583-589.
7. Brede, M., Wiesmann, F., Jahns, R., Hadamek, K., Amolt, C., Neubauer, S., Lohse, M. J. & Hein, L. (2002) Circ. 106, 2491-2496.
8. Hein, L., Altman, J. D. & Kobilka, B. K. (1999) Nature 402, 181-184.
9. Bristow, M. R. (1998) Lancet 352, SI8-SI14.
10. Liggett, S. B. (2001) Nature Medicine 7, 281-283.
11. Scheinin, M., Sallinen, J. & Haapalinna, A. (2001) Life Sci. 68, 2277-2285.
12. Small, K. M., Forbes, S. L., Rahman, F. F., Bridges, K. M. & Liggett, S. B. (2000) J. Biol. Chem. 275, 23059-23064.
13.
14. Stephens, M., Smith, N. J. & Donnelly, P. (2001) Am. J. Hum. Genet. 68, 978-989.
15. LaQuaglia, M. P., Kopp, E. B., Spengler, B. A., Meyers, M. B. & Biedler, J. L. (1991) J. Pediatr. Surg. 26, 1107-1112.
16. Rezaiguia, S., Garat, C., Delclaux, C., Meignan, M., Fleury, J., Legrand, M. A., Matthay, M. A. & Jayr, C. (1997) J. Clin. Invest. 99, 325-335.
17. Hill, W. G. & Robertson, A. (1968) Theor Appl Genet 38, 226-231.
18. Ke, X. & Cardon, L. R. (2003) Bioinformatics 19, 287-288.
19. Stephens, J. C., Schneider, J. A., Tanguay, D. A., Choi, J., Acharya, T., Stanley, S. E., Jiang, R., Messer, C. J., Chew, A., Han, J.-H. et al. (2001) Science 293, 489-493.
20. Small, K. M., Tanguay, D. A., Nandabalan, K., Zhan, P., Stephens, J. C. & Liggeft, S. B. (2003) Am. J. Pharmacogenomics 3, 65-71.

1. An isolated polynucleotide comprising a sequence of an alpha 2c adrenergic receptor (α_{2C}AR) variant, wherein the sequence consists of one of SEQ ID NOS:1-30, or a complement thereof.

2. The isolated polynucleotide of claim 1, wherein said sequence is 95% homologous to one of SEQ ID NOS:1-30, or a complement thereof.

3. An isolated polynucleotide comprising a sequence of an alpha 2c adrenergic receptor variant, or a complement thereof, wherein the sequence comprises at least one nucleotide variation located at an alpha 2c AR polymorphic site, wherein an alpha 2c AR polymorphic site is nucleotide 59, 222, 281, 358, 569, 574, 712, 946, 1125, 1376, 1673, 1698, 1705, 1942, 2397, 2408, 3570, 3633, 3804, 4110-4130, 4123, or 4394.

4. The polynucleotide of claim 3, comprising 2, 3, 4, 5, 6, 7, 8, 9, 10, 11, 12, 13, 14, 15, 16, 17, 18, 19, 20, 21, or 22 nucleotide variations.

5. The polynucleotide of claim 3, further comprising a deletion of alpha 2c AR nucleotides 3601-3612.

6. An oligonucleotide for detecting the polynucleotide of claim 3.

7. The oligonucleotide of claim 6, comprising a sequence selected from the group consisting of SEQ ID NOS:52-97.

8. A vector comprising the polynucleotide of claim 1.

9. An isolated polypeptide encoded by the polynucleotide of claim 1.

10. A cell comprising the polynucleotide of claim 1.

11. A transgenic animal comprising the polynucleotide of claim 1.

12. A kit comprising at least one oligonucleotide for detecting at least one alpha 2c AR polymorphic site and instructions for use.

13. The kit of claim 12, further comprising at least one oligonucleotide for detecting a polymorphism at alpha 2c AR nucleotides 3601-3612.

14. A method for detecting at least one alpha 2c AR polymorphic site.

15. The method of claim 14, further comprising detecting a polymorphism at alpha 2c AR nucleotides 3601-3612.

16. A method for genotyping an individual comprising the steps of a) obtaining at least one sample from the individual; b) detecting at least one alpha 2c AR polymorphic site in the sample and c) comparing the identity of the at least one polymorphic site with a known data set.

17. The method of claim of 16, further comprising detecting a polymorphism at alpha 2c AR nucleotides 3601-3612.

18. The method of claim 16, further comprising determining whether an individual is predisposed to a condition associated with an alpha2C haplotype.

19. The method of claim 16, further comprising determining whether an individual is suffering from a condition associated with an alpha2C haplotype.

20. The method of claim 16, further comprising selecting an appropriate treatment regimen.

21. The method of claim 18, wherein said condition associated with an alpha2C haplotype is heart disease.

22. The polynucleotide of claim 4, further comprising a deletion of alpha 2c AR nucleotides 3601-3612.

23. An oligonucleotide for detecting the polynucleotide of claim 4.

24. A vector comprising the polynucleotide of claim 3.

25. An isolated polypeptide encoded by the polynucleotide of claim 3.

26. A cell comprising the polynucleotide of claim 3.

27. A transgenic animal comprising the polynucleotide of claim 3.

28. The method of claim 19, wherein said condition associated with an alpha2C haplotype is heart disease.

29. The method of claim 20, wherein said condition associated with an alpha2C haplotype is heart disease.


SEQUENCE LISTING (98 sequences)

SEQ ID NO: 1 - DNA, 4625 bp, Artificial Sequence
  ALPHA2CAR HAPLOTYPE 1
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 2 - DNA, 4625 bp, Artificial Sequence
  ALPHA2CAR HAPLOTYPE 1A
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcactgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 3 - DNA, 4625 bp, Artificial Sequence
  ALPHA2CAR HAPLOTYPE 2
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcactgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 4 - DNA, 4625 bp, Artificial Sequence
  ALPHA2CAR HAPLOTYPE 2A
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 5 - DNA, 4613 bp, Artificial Sequence
  ALPHA2CAR HAPLOTYPE 3
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 6 - DNA, 4613 bp, Artificial Sequence
  ALPHA2CAR HAPLOTYPE 4
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctcggggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 7 - DNA, 4613 bp, Artificial Sequence
  ALPHA2CAR HAPLOTYPE 4A
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctcggggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcactgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 8 - DNA, 4625 bp, Artificial Sequence
  ALPHA2CAR HAPLOTYPE 5
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcgcccctcactctctgccatcgcagagggctcaatagagacaagggctccagcgggccgatcatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaacgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttacaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 9 - DNA, 4613 bp, Artificial Sequence
  ALPHA2CAR HAPLOTYPE 6
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 10 - DNA, 4613 bp, Artificial Sequence
  ALPHA2CAR HAPLOTYPE 6A
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 11 - DNA, 4613 bp, Artificial Sequence
  ALPHA2CAR HAPLOTYPE 7
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttagtcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 12 - DNA, 4604 bp, Artificial Sequence
  ALPHA2CAR HAPLOTYPE 8
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccctggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccacccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 13 - DNA, 4604 bp, Artificial Sequence
  ALPHA2CAR HAPLOTYPE 8A
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccctggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccacccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcactgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 14 - DNA, 4613 bp, Artificial Sequence
  ALPHA2CAR HAPLOTYPE 9
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcccgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 15 - DNA, 4604 bp, Artificial Sequence
  ALPHA2CAR HAPLOTYPE 10
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 16 - DNA, 4625 bp, Artificial Sequence
  ALPHA2CAR HAPLOTYPE 11
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 17 - DNA, 4625 bp, Artificial Sequence
  ALPHA2CAR HAPLOTYPE 12
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcgcccctcactctctgccatcgcagagggctcaatagagacaagggctccagcgggccgatcatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttacaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 18 - DNA, 4625 bp, Artificial Sequence
  ALPHA2CAR HAPLOTYPE 13
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacagctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 19 - DNA, 4625 bp, Artificial Sequence
  ALPHA2CAR HAPLOTYPE 14
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcgggcgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcactgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 20 - DNA, 4613 bp, Artificial Sequence
  ALPHA2CAR HAPLOTYPE 15
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 21 - DNA, 4625 bp, Artificial Sequence
  ALPHA2CAR HAPLOTYPE 16
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcactgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 22 - DNA, 4604 bp, Artificial Sequence
  ALPHA2CAR HAPLOTYPE 17
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 23 - DNA, 4613 bp, Artificial Sequence
  ALPHA2CAR HAPLOTYPE 18
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaagctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggacggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 24 - DNA, 4592 bp, Artificial Sequence
  ALPHA2CAR HAPLOTYPE 19
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcccgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 25 - DNA, 4613 bp, Artificial Sequence
  ALPHA2CAR HAPLOTYPE 20
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaagctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacggttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggacggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgccgaggggggcgcgggcggtgcggacgggcagggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggtgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 26 - DNA, 4604 bp, Artificial Sequence
  ALPHA2CAR HAPLOTYPE 21
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccctggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccacccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcccgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 27 - DNA, 4604 bp, Artificial Sequence
  ALPHA2CAR HAPLOTYPE 22
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccctggctcggcctccacacccccaatgcaggccctgctggtgacaaacatggtgaagaacgaagccgtagctcctggcagtgtctctgcggagacagacatgctccccagtagggctgagcagcagagaacacgacaggctctgaagctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccacccaggacggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 28 - DNA, 4604 bp, Artificial Sequence
  ALPHA2CAR HAPLOTYPE 23
tcccactggaggaaggacagcctacagaggcctcccccttcctcataacacctcctcactctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccctggcttggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccacccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcctgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 29 - DNA, 4625 bp, Artificial Sequence
  ALPHA2CAR HAPLOTYPE 24
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcccgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctttcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaagacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 30 - DNA, 4625 bp, Artificial Sequence
  ALPHA2CAR HAPLOTYPE 25
tcccactggaggaaggacagcctacagaggcctcccccttcctcataacacctcctcattctctaagcacagtttggtcacctcctccaggaaggcttccttgacaaccccgctcccaggctgggggaggagccccagggcactgatggttgtcagcgcattcacctcagtgggtggccccatccacccacagatggtgagcacctcaactgaggccaggcccccctcactctctgccatcgcagagggctcaatagagacaagggctccagcgggccgaccatgaaggaagcaggtgtgtgtgggcttccctgaaaaatgcacctccaggtgccctggacaggtggcaccttttaggcaaccagaaccaggagggcctttagtaaggatggtgaccctccaccatccccaggtcacacacctcccacaggacagccatggcctgttgcctccccgaagggcatatgtgccctagactagcacaggactggcacagaaggctttggctccaacgtgcgcccctgcttggcaggcccatcatctgaaatatttgctttctgccgggccccggctcggcctccacacccccaatgcaggccctgctggtgacaaacatggtgaagaacgaagccgtagctcctggcagtgtctctgcggagacagacatgctccccagtagggctgagcagcagagaacacgacaggctctgaggctaaagctggcgcccttcaggagctcagagagaagctgccccaggccacgggcatggataggcatcccgggggtagggcagtcactccagccgctcttcatgatgacctcccgggcactcagaagcgccccttccccgccggttctagccacaggatggacagaccttccctccctctctcctgcgggcccatgcagggagaggccagccagctggggttcgtttctggacgtttgggagaagggtcgtcccttcggtcctagcagagaaacgtcaggcctgagaactccagctctcctcctggatcacctcgtccctggaagtccctctcccacagaccccaggctgcctggaggcagaagccagcacccaacatgggccacagagtggcagtgcacgtggcagtgcacatctgcagaatggaggtgcgcggacgggggcgagggtgcacgggggctacctcaacggccgctctcccctgagcccacccttgcaaaaagctccatcatccaggcagctccgttctgggtgtttgtcagaggtggtgactttgaacacctctccatcctttaaaagcagggcctgagagcctaaggattaaacttcaatcactgctaacatgggtgcagagcttgggagcttgcccagggcttccacagacaccgccgttagttatgattcatgctccagcagcgctgggaagtgggccgagggaactgaggccggggtgggtgcggaggtggctctgacggggagcgccgatccaggatctggtccggaccctgagctcggcaggacctgtgcgcacgcaggtagcgtcgcgtcctcccccaggctgcgagaggttgctctgccatcaggccatggaccccgagccgcccgtgctgcgcgcgcgcgtgtgcgcgcccccgcctgcgcccatgcctgcgccgggggaggcgaaggaggctccagtcttagaaagagcagcttctggaactcaccgcccaggccggccgccgctcggccccgtcccgcaggctgcaggcggccctggagggggcgccctcgccgagcgcgcgccccgcgccgccgccccggactcctccccggcgccgcgcgggcaggttcgaccaggcggccgcgggctccggttcccggccagctccccagggcccgcggcccgccccgccccgcgcgcccgccccgctgcgcaactcgacccaagttggaagccgatcgcaggcggccgcactcgcgcccagcgcagggcggcggcggcggcggcggcgcagctccggcgagcgaggcggcggccgcacggccaagcgtggaccgcggggggcgcccgcgcccgggagcagccggaggactcgcggcggcgccggcgccccgcccgggaaagtaaagttggagacggagggagcgcgcgggcgcgggggcccggaggagcggcggccggcgccccggcgcgccgagccctagccggccggatgggaggcggagcgcccgggccgccgccgcctgtcgcctgcgccccggctgggctccgggaccgcggggccgctcaggcacgcccgctcgggcgccgcgtcgccgtgggctcgccgccggggcgctccccgtgagccgggccgagggcggggccgcgcgaggaccccgggacctgcccccctccccccgcagccgcgtcgccgctcgctccgggcgcctcccgctctgcacttacacgctcggcagctgcggggagcccggcagccacgctctccggcgcgccgcccgcggagccaccacggccgagggccggctgctgggcgccgcggtccccggcgggcgcgcccgagcagcaggcggcgatgcgggcgccgaccccggctggggggcgcccgagctgccgcggctgcgccccggctccaggagggacggcgtagctcgcgggaggaccatggcgtccccggcgctggcggcggcgctggcggtggcggcagcggcgggccccaatgcgagcggcgcgggcgagaggggcagcggcggggttgccaatgcctcgggggcttcctgggggccgccgcgcggccagtactcggcgggcgcggtggcagggctggctgccgtggtgggcttcctcatcgtcttcaccgtggtgggcaacgtgctggtggtgatcgccgtgctgaccagccgggcgctgcgcgcgccacagaacctcttcctggtgtcgctggcctcggccgacatcctggtggccacgctggtcatgcccttctcgttggccaacgagctcatggcctactggtacttcgggcaggtgtggtgcggcgtgtacctggcgctcgatgtgctgttttgcacctcgtcgatcgtgcatctgtgtgccatcagcctggaccgctactggtcggtgacgcaggccgtcgagtacaacctgaagcgcacaccacgccgcgtcaaggccaccatcgtggccgtgtggctcatctcggccgtcatctccttcccgccgctggtctcgctctaccgccagcccgacggcgccgcctacccgcagtgcggcctcaacgacgagacctggtacatcctgtcctcctgcatcggctccttcttcgcgccctgcctcatcatgggcctggtctacgcgcgcatctaccgagtggccaagctgcgcacgcgcacgctcagcgagaagcgcgcccccgtgggccccgacggtgcgtccccgactaccgaaaacgggctgggcgcggcggcaggcgcaggcgagaacgggcactgcgcgcccccgcccgccgacgtggagccggacgagagcagcgcagcggccgagaggcggcggcgccggggcgcgttgcggcggggcgggcggcggcgagcgggcgcggaggggggcgcgggcggtgcggacgggcagggggcggggccgggggcggctgagtcgggggcgctgaccgcctccaggtccccggggcccggtggccgcctgtcgcgcgccagctcgcgctccgtcgagttcttcctgtcgcgccggcgccgggcgcgcagcagcgtgtgccgccgcaaggtggcccaggcgcgcgagaagcgcttcacctttgtgctggctgtggtcatgggcgtgttcgtgctctgctggttccccttcttcttcagctacagcctgtacggcatctgccgcgaggcctgccaggtgcccggcccgctcttcaagttcttcttctggatcggctactgcaacagctcgctcaacccggtcatctacacggtcttcaaccaggatttccggcgatcctttaagcacatcctcttccgacggaggagaaggggcttcaggcagtgactcgcacccgtctgggaatcctggacagctccgcgctcggggctgggcagaaggggcggcccggacgggggagctttcccagagacccggggagctctcccagagacccggggatggattggcctccagggcgcaggggagggtgcggcagggcaggagcttggcagagagatagccgggctccagggagtggggaggagagagggggagacccctttgccttcccccctcagcaaggggctgcttctggggctccctgcctggatccagctctgggagccctgccgaggtgtggctgtgaggtcagggttttagagagcagtggcagaggtagccccctaaatgggcaagcaaggagccccccaaacacactaccactccccatccccgtctgaccaagggctgacttctccaggacctagtcggggggtggctgccagggggcaaggagaaagcaccgacaatctttgattactgaaagtatttaaatgtttgccaaaaacaacagccaaaacaaccaaactattttctaaataaacctttgtaatctaatgttgggtgccgcagtcagtccttgagcttgggggctgggggtatct

SEQ ID NO: 31 - DNA, 4625 bp, Artificial Sequence; identical to SEQ ID NO: 1
  ALPHA2C Adrenergic Receptor

SEQ ID NO: 32 - DNA, 25 bp, Artificial Sequence
  Primer for promoter region fragment 1
tcccactggaggaaggacagcctac

SEQ ID NO: 33 - DNA, 24 bp, Artificial Sequence
  Primer for promoter region fragment 1
cacgttggagccaaagccttctgt

SEQ ID NO: 34 - DNA, 20 bp, Artificial Sequence
  Primer for promoter region fragment 2
tttagtaaggatggtgaccc

SEQ ID NO: 35 - DNA, 24 bp, Artificial Sequence
  Primer for promoter region fragment 2
ttctcccaaacgtccagaaacgaa

SEQ ID NO: 36 - DNA, 24 bp, Artificial Sequence
  Primer for promoter region fragment 3
cagccgctcttcatgatgacctcc

SEQ ID NO: 37 - DNA, 24 bp, Artificial Sequence
  Primer for promoter region fragment 3
gctgctggagcatgaatcataact

SEQ ID NO: 38 - DNA, 24 bp, Artificial Sequence
  Primer for promoter region fragment 4
acttcaatcactgctaacatgggt

SEQ ID NO: 39 - DNA, 24 bp, Artificial Sequence
  Primer for promoter region fragment 4
gagcccgcggccgcctggtcgaac

SEQ ID NO: 40 - DNA, 26 bp, Artificial Sequence
  Primer for 5′UTR region fragment 5
cttagaaagagcagcttctggaactc

SEQ ID NO: 41 - DNA, 24 bp, Artificial Sequence
  Primer for 5′UTR region fragment 5
ctgccgagcgtgtaagtgcagagc

SEQ ID NO: 42 - DNA, 24 bp, Artificial Sequence
  Primer for 5′UTR region fragment 6
ggaaagtaaagttggagacggagg

SEQ ID NO: 43 - DNA, 25 bp, Artificial Sequence
  Primer for 5′UTR region fragment 6
acggtgaagacgatgaggaagccca

SEQ ID NO: 44 - DNA, 27 bp, Artificial Sequence
  Primer for Coding region fragment 7
tagctcgcgggaggaccatggcgtccc

SEQ ID NO: 45 - DNA, 29 bp, Artificial Sequence
  Primer for Coding region fragment 7
gatgcaggaggacaggatgtaccaggtct

SEQ ID NO: 46 - DNA, 24 bp, Artificial Sequence
  Primer for Coding region fragment 8
ccatcgtcgccgtgtggctcatct

SEQ ID NO: 47 - DNA, 24 bp, Artificial Sequence
  Primer for Coding region fragment 8
aggcctcgcggcagatgccgtaca

SEQ ID NO: 48 - DNA, 22 bp, Artificial Sequence
  Primer for Coding region fragment 9
gagaagcgcttcacctttgtgc

SEQ ID NO: 49 - DNA, 21 bp, Artificial Sequence
  Primer for Coding region fragment 9
gccctggaggccaatccatcc

SEQ ID NO: 50 - DNA, 24 bp, Artificial Sequence
  Primer for 3′UTR region fragment 10
ccgacggaggagaaggggcttcag

SEQ ID NO: 51 - DNA, 25 bp, Artificial Sequence
  Primer for 3′UTR region fragment 10
aactcgaacccccgacccccataga

SEQ ID NO: 52 - DNA, 30 bp, Artificial Sequence
  Primer for Location Code a
ggcctcccccttcctcataacacctcctga

SEQ ID NO: 53 - DNA, 24 bp, Artificial Sequence
  Primer for Location Code a
acacacctgcttccttcatggtcg

SEQ ID NO: 54 - DNA, 25 bp, Artificial Sequence
  Primer for Location Code b
tcccactggaggaaggacagccatc

SEQ ID NO: 55 - DNA, 24 bp, Artificial Sequence
  Primer for Location Code b
cacgttggagccaaagccttctgt

SEQ ID NO: 56 - DNA, 25 bp, Artificial Sequence
  Primer for Location Code c
tcccactggaggaaggacagccatc

SEQ ID NO: 57 - DNA, 24 bp, Artificial Sequence
  Primer for Location Code c
cacgttggagccaaagccttctgt

SEQ ID NO: 58 - DNA, 25 bp, Artificial Sequence
  Primer for Location Code d
tcccactggaggaaggacagccatc

SEQ ID NO: 59 - DNA, 24 bp, Artificial Sequence
  Primer for Location Code d
cacgttggagccaaagccttctgt

SEQ ID NO: 60 - DNA, 19 bp, Artificial Sequence
  Primer for Location Code e
ttagtaaggatggtgaccc

SEQ ID NO: 61 - DNA, 21 bp, Artificial Sequence
  Primer for Location Code e
cttcgttcttcaccatgtttg

SEQ ID NO: 62 - DNA, 31 bp, Artificial Sequence
  Primer for Location Code f
tgaaatatttgctttctgccgggccccggat

SEQ ID NO: 63 - DNA, 22 bp, Artificial Sequence
  Primer for Location Code f
tctgagctcctgaagggcgcca

SEQ ID NO: 64 - DNA, 24 bp, Artificial Sequence
  Primer for Location Code g
aggcccatcatctgaaatatttgc

SEQ ID NO: 65 - DNA, 22 bp, Artificial Sequence
  Primer for Location Code g
tctgagctcctgaagggcgcca

SEQ ID NO: 66 - DNA, 24 bp, Artificial Sequence
  Primer for Location Code h
cagccgctcttcatgatgacctcc

SEQ ID NO: 67 - DNA, 24 bp, Artificial Sequence
  Primer for Location Code h
gctgctggagcatgaatcataact

SEQ ID NO: 68 - DNA, 24 bp, Artificial Sequence
  Primer for Location Code i
cagccgctcttcatgatgacctcc

SEQ ID NO: 69 - DNA, 24 bp, Artificial Sequence
  Primer for Location Code i
gctgctggagcatgaatcataact

SEQ ID NO: 70 - DNA, 24 bp, Artificial Sequence
  Primer for Location Code j
acttcaatcactgctaacatgggt

SEQ ID NO: 71 - DNA, 23 bp, Artificial Sequence
  Primer for Location Code j
tgatggcagagcaacctctcgca

SEQ ID NO: 72 - DNA, 24 bp, Artificial Sequence
  Primer for Location Code k
acttcaatcactgctaacatgggt

SEQ ID NO: 73 - DNA, 24 bp, Artificial Sequence
  Primer for Location Code k
gagcccgcggccgcctggtcgaac

SEQ ID NO: 74 - DNA, 31 bp, Artificial Sequence
  Primer for Location Code l
agtcttaaaagagcagcttctggaactctcc

SEQ ID NO: 75 - DNA, 24 bp, Artificial Sequence
  Primer for Location Code l
gagcccgcggccgcctggtcgaac

SEQ ID NO: 76 - DNA, 24 bp, Artificial Sequence
  Primer for Location Code m
acttcaatcactgctaacatgggt

SEQ ID NO: 77 - DNA, 23 bp, Artificial Sequence
  Primer for Location Code m
gagcccgcggccgcctggtcgaa

SEQ ID NO: 78 - DNA, 26 bp, Artificial Sequence
  Primer for Location Code n
cttagaaagagcagcttctggaactc

SEQ ID NO: 79 - DNA, 24 bp, Artificial Sequence
  Primer for Location Code n
ctgccgagcgtgtaagtgcagagc

SEQ ID NO: 80 - DNA, 24 bp, Artificial Sequence
  Primer for Location Code o
gagccctagccggccggatgggag

SEQ ID NO: 81 - DNA, 23 bp, Artificial Sequence
  Primer for Location Code o
cccagcagccggccctcggccgt

SEQ ID NO: 82 - DNA, 23 bp, Artificial Sequence
  Primer for Location Code p
gtcgccgctcgctccgggcgcct

SEQ ID NO: 83 - DNA, 23 bp, Artificial Sequence
  Primer for Location Code p
agcgccggggacgccatggtcct

SEQ ID NO: 84 - DNA, 20 bp, Artificial Sequence
  Primer for Location Code q
agccggacgagagcagcgca

SEQ ID NO: 85 - DNA, 24 bp, Artificial Sequence
  Primer for Location Code q
aggcctcgcggcagatgccgtaca

SEQ ID NO: 86 - DNA, 24 bp, Artificial Sequence
  Primer for Location Code r
ccgacggaggagaaggggcttcag

SEQ ID NO: 87 - DNA, 21 bp, Artificial Sequence
  Primer for Location Code r
gccctggaggccaatccatcc

SEQ ID NO: 88 - DNA, 22 bp, Artificial Sequence
  Primer for Location Code s
gagaagcgcttcacctttgtgc

SEQ ID NO: 89 - DNA, 17 bp, Artificial Sequence
  Primer for Location Code s
gccctggaggccaatcc

SEQ ID NO: 90 - DNA, 28 bp, Artificial Sequence
  Primer for Location Code t
gggcaggagcttggcagagagatagccg

SEQ ID NO: 91 - DNA, 33 bp, Artificial Sequence
  Primer for Location Code t
ccttggtcagacggggatggggagtggtagagt

SEQ ID NO: 92 - DNA, 20 bp, Artificial Sequence
  Primer for Location Code u
agccggacgagagcagcgca

SEQ ID NO: 93 - DNA, 24 bp, Artificial Sequence
  Primer for Location Code u
aggcctcgcggcagatgccgtaca

SEQ ID NO: 94 - DNA, 20 bp, Artificial Sequence
  Primer for Location Code v
agccggacgagagcagcgca

SEQ ID NO: 95 - DNA, 24 bp, Artificial Sequence
  Primer for Location Code v
aggcctcgcggcagatgccgtaca

SEQ ID NO: 96 - DNA, 33 bp, Artificial Sequence
  Primer for Location Code w
cgcttcacctttgtgctggctgtggtcatgcgc

SEQ ID NO: 97 - DNA, 22 bp, Artificial Sequence
  Primer for Location Code w
gtgtagatgaccgggttgagca

SEQ ID NO: 98 - protein, 462 aa, Artificial Sequence
  alpha2C adrenergic receptor
MASPALAAALAVAAAAGPNASGAGERGSGGVANASGASWGPPRGQYSAGAVAGLAAVVGFLIVFTVVGNVLVVIAVLTSRALRAPQNLFLVSLASADILVATLVMPFSLANELMAYWYFGQVWCGVYLALDVLFCTSSIVHLCAISLDRYWSVTQAVEYNLKRTPRRVKATIVAVWLISAVISFPPLVSLYRQPDGAAYPQCGLNDETWYILSSCIGSFFAPCLIMGLVYARIYRVAKLRTRTLSEKRAPVGPDGASPTTENGLGAAAGAGENGHCAPPPADVEPDESSAAAERRRRRGALRRGGRRRAGAEGGAGGADGQGAGPGAAESGALTASRSPGPGGRLSRASSRSVEFFLSRRRRARSSVCRRKVAQAREKRFTFVLAVVMGVFVLCWFPFFFSYSLYGICREACQVPGPLFKFFFWIGYCNSSLNPVIYTVFNQDFRRSFKHILFRRRRRGFRQ